SEQUENCE LISTING (3 sequences)

SEQ ID NO: 1 - protein, 16 aa, Artificial Sequence
  An enzymatically degradable (Deg) peptide
GCNSVPMSMRGGSNCG

SEQ ID NO: 2 - protein, 8 aa, Artificial Sequence
  A peptide sequence that cleaves in the presence of cell-secreted matrix metallopeptidases (MMPs)
VPMSMRGG

SEQ ID NO: 3 - protein, 16 aa, Artificial Sequence
  A non-enzymatically degradable (NonDeg) crosslinker peptide
GCHGNSGGSGGNEECG

(19) United States
(12) Patent Application Publication
(10) Pub. No.: US 2024/0050626 A1
(43) Pub. Date: Feb. 15, 2024

(54) Synthetic Aligned Tissue Grafts and Methods of Using the Same

(71) Applicant: Rowan University, Glassboro, NJ (US)

(72) Inventors: Vince Beachley, Medford Lakes, NJ (US); Sebastian L. Vega, Sewell, NJ (US); Dave Joa, Glassboro, NJ (US)

(21) Appl. No.: 18/251,166

(22) PCT Filed: May 27, 2022
(86) PCT No.: PCT/US2022/031429

§ 371 Date: Apr. 28, 2023

Related U.S. Application Data
(60) Provisional application No. 63/194,316, filed on May 28, 2021.

(51) Int. Cl. A61L 27/48; A61L 27/52; A61L 27/58

(52) U.S. Cl. CPC A61L 27/48; A61L 27/52; A61L 27/58; A61L 2400/12; A61L 2430/32

(57) ABSTRACT

The present disclosure relates to a multi-layer composite comprising at least a first sheet and a second sheet of aligned nanofibers coated with a biodegradable hydrogel, wherein the at least first and second sheets of aligned coated nanofibers are stacked directly on top of each other and a portion of the biodegradable hydrogel coating on the first sheet is mixed and crosslinked with a portion of the biodegradable hydrogel coating on the second sheet. In another aspect, the present disclosure relates to a method of regenerating an aligned soft tissue in a subject, the method comprising surgically implanting a synthetic tissue graft comprising the multi-layer composite in the subject at a site of missing or injured tissue. In yet another aspect, the present disclosure relates to a synthetic nerve graft comprising the multi-layer composite.

### CROSS-REFERENCE TO RELATED APPLICATIONS

The present application claims priority under 35 U.S.C. § 119(e) to U.S. Provisional Patent Application No. 63/194,316, filed May 28, 2021, which is incorporated herein by reference in its entirety.

### STATEMENT REGARDING FEDERALLY SPONSORED RESEARCH OR DEVELOPMENT

This invention was made with government support under W911NF-17-2-0227 awarded by the Department of Defense. The government has certain rights in the invention.

### BACKGROUND

Peripheral nerve injuries can result from over-stretching, lacerations, or compression, and affect the brain's ability to communicate with muscles and organs. Most peripheral nerve injuries occur from trauma and affect the upper limbs, and although many lower grade nerve injuries heal without intervention, higher degree injuries have a poor prognosis. Once a nerve sustains a high degree injury, the distal nerve segment is unable to effectively transmit signals to neuromuscular junctions, leading to muscular atrophy and permanent damage to distal tissues. Severe peripheral nerve injuries have a significant impact on quality of life, resulting in neuropathic pain or complete paralysis of the affected limb. Peripheral nerve injuries are common, affecting over 20 million people in the United States alone. These injuries are also a significant financial burden, incurring over $150 billion spent in annual healthcare dollars.

Nerve grafting is a surgical intervention in which a segment of unrelated nerve is used to bridge an injured or missing portion of nerve. Nerve grafts date as far back as 1896, and although advances have been made since, a high percentage of patients are still left with motor and sensory deficits. Despite the limitations of nerve grafts, nerve autografting is the standard of care for severe peripheral nerve injuries. Due to the limited supply of donor autograft nerves and the morbidity of harvesting these nerves, there has been increased interest in nerve allografting. A direct comparison between nerve autografts and allografts in a rat sciatic nerve gap model showed no differences in functional outcome, and this has also been seen with patients treated with nerve allografts. However, in addition to poor functional outcomes, similar to autografts, allografts are costly, have an associated risk of disease transmission, and raise concerns for immunological host rejection.

Due to the limitations of nerve autografts and allografts, recent efforts have relied on creating biomaterial-based nerve grafts to bridge nerve gaps in severe peripheral nerve injuries. Hollow guidance tubes can facilitate the accumulation of neurotrophic factors and promote axon regeneration, while reducing myofibroblast infiltration and scar formation. Despite successes in biomaterials-based nerve conduits, they have shown only partial improvement in functional outcomes and are limited to nerve gaps of 3 cm or less. Due to this limitation, currently marketed synthetic nerve graft are only available in lengths of 1-3 cm. A key question in synthetic peripheral nerve repair is whether effective regeneration for defect lengths greater than 3 cm is possible.

Utilizing aligned nanofibers in the grafts enhances the alignment and elongation of the cells and may permit the development of synthetic nerve grafts with lengths greater than 3 cm. However, aligned nanofibers are formed and used as components of only 2D films, which are not appropriate for 3D tissue regeneration. The 2D films do not have the proper geometry to act as they would in the body, and dense fiber packing in the films blocks cell infiltration.

There is a need in the art for synthetic tissue grafts that promote regeneration of aligned soft tissues in a defined direction. There is also a need in the art for methods of making and using synthetic tissue grafts that promote the regeneration of aligned soft tissues in a defined direction. The present disclosure satisfies these unmet needs.

### SUMMARY

In some aspects, the present invention is directed to a method of regenerating an aligned soft tissue in a subject.

In some embodiments, the method includes surgically implanting a synthetic tissue graft in the subject at a site of missing or injured soft tissue.

In some embodiments, the synthetic tissue graft is a multi-layer composite includes at least a first and a second sheets of aligned nanofibers coated with a biodegradable hydrogel.

In some embodiments, the at least first and second sheets of coated aligned nanofibers are stacked directly on top of each other.

In some embodiments, a portion of the biodegradable hydrogel coating on the first sheet is mixed and crosslinked with a portion of the biodegradable hydrogel coating on the second sheet.

In some embodiments, the aligned soft tissue is a nerve and the synthetic tissue graft is a synthetic nerve graft.

In some embodiments, the synthetic nerve graft includes the multi-layer composite formed into a cylinder shape, wherein the outside surface of the cylinder is covered with an outer conduit.

In some embodiments, each of the at least first and second sheets of coated aligned nanofibers independently include substantially aligned individual nanofibers having a controlled diameter and spacing.

In some embodiments, each of the at least first and second sheets of coated aligned nanofibers independently include substantially aligned individual nanofibers having a length of about 0.25 cm to about 30 cm.

In some embodiments, each of the at least first and second sheets of coated aligned nanofibers independently include substantially aligned individual nanofibers that are covalently bonded to the biodegradable hydrogel coating.

In some embodiments, the at least first and second sheets of coated aligned nanofibers include substantially aligned individual polycaprolactone, alginate, polyacrylonitrile, or poly(lactic acid) nanofibers.

In some embodiments, each of the at least first and second sheets of aligned nanofibers coated with a biodegradable hydrogel has a controlled thickness and the multi-layer composite has a defined three dimensional structure.

In some embodiments, the biodegradable hydrogel coating include gelatin, poly(ethylene glycol), hyaluronic acid, collagen, polyacrylamide, alginate, or chemically modified versions thereof.

In some embodiments, the biodegradable hydrogel coating includes gelatin or chemically modified versions thereof.

In some embodiments, cells at the site of the missing or injured tissue degrade the biodegradable hydrogel and grow into the multi-layer composite in the direction of the at least first and second sheets of coated aligned nanofibers, thus regenerating the aligned soft tissue in the subject.

In some embodiments, the biodegradable hydrogel coating is reversibly or irreversibly gelled or cured before, during or after layer stacking.

In some embodiments, the multi-layer composite includes one or more additional sheets of aligned nanofibers coated with a biodegradable hydrogel such that the multi-layer composite includes three or more sheets of coated aligned nanofibers stacked on top of each other.

In some embodiments, each coated sheet of aligned nanofibers completely covers an adjacent sheet of coated aligned nanofibers.

In some embodiments, a portion of the biodegradable hydrogel coating on each sheet of coated aligned nanofibers is mixed and crosslinked with a portion of the biodegradable hydrogel coating on directly adjacent coated sheets of aligned nanofibers.

In some embodiments, the method further includes forming a gradient of a chemical modification on the aligned nanofibers or in the coated biodegradable hydrogel in the direction of the nanofibers.

In some embodiments, the chemical modification includes a peptide tethered to a surface of the nanofibers or to a molecular backbone of the biodegradable hydrogel, or a degradable crosslinker in the biodegradable hydrogel.

In some embodiments, the present invention is directed to a synthetic nerve graft.

In some embodiments, the synthetic nerve graft includes a multi-layer composite.

In some embodiments, the multi-layer composite includes at least a first and second sheets of aligned nanofibers coated with a biodegradable hydrogel.

In some embodiments, the at least first and second sheets of coated aligned nanofibers are stacked directly on top of each other.

In some embodiments, a portion of the biodegradable hydrogel coating on the first sheet is mixed and crosslinked with a portion of the biodegradable hydrogel coating on the second sheet.

In some embodiments, the synthetic nerve graft further includes an outer conduit covering the outside surface of a cylinder-shaped multi-layer composite.

In some embodiments, each of the at least first and second sheets of coated aligned nanofibers independently include substantially aligned individual nanofibers having a controlled diameter and spacing.

In some embodiments, each of the at least first and second sheets of coated aligned nanofibers independently include substantially aligned individual nanofibers having a length of about 0.25 cm to about 30 cm.

In some embodiments, each of the at least first and second sheets of coated aligned nanofibers independently include substantially aligned individual nanofibers that are covalently bonded to the biodegradable hydrogel coating.

In some embodiments, the at least first and second sheets of coated aligned nanofibers include substantially aligned individual polycaprolactone, alginate, polyacrylonitrile, or poly(lactic acid) nanofibers.

In some embodiments, each of the at least first and second sheets of aligned nanofibers coated with a biodegradable hydrogel has a controlled thickness and the multi-layer composite has a defined three dimensional structure.

In some embodiments, the biodegradable hydrogel coating includes gelatin, poly(ethylene glycol), hyaluronic acid, collagen, polyacrylamide, alginate, or chemically modified versions thereof.

In some embodiments, the biodegradable hydrogel coating includes gelatin or chemically modified versions thereof.

In some embodiments, the multi-layer composite includes one or more additional sheets of aligned nanofibers coated with a biodegradable hydrogel such that the multi-layer composite includes three or more sheets of coated aligned nanofibers stacked on top of each other.

In some embodiments, each coated sheet of aligned nanofibers completely covers an adjacent sheet of coated aligned nanofibers.

In some embodiments, a portion of the biodegradable hydrogel coating on each sheet of coated aligned nanofibers is mixed and crosslinked with a portion of the biodegradable hydrogel coating on directly adjacent coated sheets of aligned nanofibers.

In some embodiments, the synthetic nerve graft bridges a nerve gap in a subject and promotes neuron alignment and axon elongation across the nerve gap.

In some embodiments, the aligned nanofibers or the coated biodegradable hydrogel has a gradient of a chemical modification in the direction of the nanofibers.

In some embodiments, the chemical modification includes a peptide tethered to a surface of the nanofibers or to a molecular backbone of thåe biodegradable hydrogel, or a degradable crosslinker in the biodegradable hydrogel.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 is a table with the input and evaluation parameters for the stability study of the nerve grafts, in accordance with some embodiments.

FIG. 2A depicts multilayered composites are formed by alternate stacking of congealed (20° C.) and liquid (37° C.) composite films, in accordance with some embodiments. The warm liquid film causes local melting of the adjacent thermo-reversible congealed films at the interface to form a continuous fusion between layers upon cooling. Subsequent UV curing results in permanent crosslinks between norbornene and thiol groups to form a solid nanofiber-hydrogel composite that remains a gel at temperatures above 37° C. FIG. 2B shows a PLLA nanofiber containing 16.6% low molecular weight thiol (SH) terminated PLLA fraction embedded in a norbornone-functionalized gelatin (GelNor) hydrogel with thiol terminated degradable peptide linkers. Heparin is shown attached to some of the surface exposed thiol groups as proposed for growth factor gradient functionalization.

FIG. 3 is a table of the input and evaluation parameters used to identify nanofiber-hydrogel composites that maximize neural cell alignment and axon growth in vitro, in accordance with some embodiments.

FIG. 4 shows in the top panel that previous attempts to incorporate aligned nanofibers into a nerve graft rely on dense nanofiber mats that block axon growth over a large percentage of the graft cross-section, in accordance with some embodiments. Axons that regrow further than 7 μm away from dense nanofiber films are unable to directly touch aligned nanofibers for contact guidance. Red boxes highlight blocked areas, Yellow boxes highlight lack of contact guidance, and Green boxes highlight areas where direct contact guidance can occur. The bottom panel contains a zoomed schematic showing a (100 μm×100 μm) patch of graft cross-section where dense nanofiber mat would block ˜60% of the cross-sectional area while providing direct axon contact guidance to only ˜15% of the cross-sectional area. A schematic of a (100 μm×100 μm) patch of proposed graft cross-section, with distributed aligned 500 nm diameter fibers supported by permissive hydrogel matrix, is predicted to provide direct contact to 99.8% of the cross-sectional area, while blocking only 0.2%.

FIG. 5A shows that automated track electrospinning continuously moves aligned and suspended individual nanofibers away from collecting area onto a removable rack, in accordance with some embodiments. FIG. 5B shows that fiber density can be controlled by adjusting collection time. FIG. 5C shows that aligned nanofiber-hydrogel composite films are easily formed by dip coating nanofiber arrays in hydrogel solution (gelatin shown). FIG. 5D shows that hydrogel macromers modified with norbornene (hyaluronic acid shown as general example) can undergo light-mediated thiol-norbornene reactions with di-thiol crosslinkers to form hydrogels.

FIG. 6A is a representative image of nanofiber layer (red) embedded in gelatin hydrogel (green), in accordance with some embodiments. FIG. 6B shows that composite thickness correlates with increasing hydrogel wt %. FIG. 6C shows that NSCs align along the direction of nanofibers and express neural biomarkers (pIII tubulin, green; nuclei, blue; nanofibers, red). FIG. 6D is a schematic of three-layer composite (PC-12s in center layer) for preliminary cell studies. FIG. 6E shows that PC-12 cells (phalloidin, green) encapsulated in 3D nanofiber-hydrogel composite (red microspheres added to visualize hydrogel volume) are able to remodel the hydrogel and align in the direction of nanofibers. FIG. 6F shows that PC-12 cells encapsulated in 3D hydrogels do not spread in the absence of nanofibers. Scale bars: C 50 μm; E, F 100 μm.

FIG. 7A provides two 15-layer scaffolds: layer thickness is controlled by dipping speed and temperature (left=37° C. & fast, right=55° C. & slow), in accordance with some embodiments. FIG. 7B is a thin longitudinal slice of a 23-layer scaffold revealing a layered fiber structure. FIG. 7C is a light microscope image of the yellow box in B showing aligned microstructure. FIG. 7D is a confocal microscopy image showing regular spacing between fiber layers. FIG. 7E depicts nanofiber-hydrogel composite segments stabilized to metal rods with sacrificial matrix and aligned nanofibers are rolled to form an outer conduit. FIG. 7F shows that GelMe hydrogels retained their shape and volume after 14 days in PBS at 4° C. FIG. 7G shows that the compressive modulus of GelMe hydrogels was not significantly reduced after 14 days in PBS at 4° C. FIG. 7H shows that a 2 mm diameter full graft with nanofiber-hydrogel core retained qualitative appearance and mechanics after 2 months in PBS.

FIG. 8A shows 1 cm segments of rat sciatic nerve that were removed and replaced with grafts, in accordance with some embodiments. FIG. 8B depicts that nanofiber-hydrogel graft explants showed good integration with nerve 3 weeks after implantation. Arrows point to conduit/nerve interface. FIG. 8C uses H&E staining to show axon growth through a nanofiber-hydrogel graft and at 4 weeks. FIG. 8D uses H&E staining to show axon growth through an allograft at 4 weeks. FIG. 8E depicts a nanofiber-hydrogel graft at 4 weeks, which contains aligned microstructure and many infiltrating cells at the graft center. FIG. 8F depicts that similar aligned microstructure and less cell infiltration was present in the allograft. Scale bar=200 μm FIG. 9A shows that the parallel automated tracks create a continuous collection of fibers during electrospinning, in accordance with some embodiments. The fibers are pulled down to a collection tray and then a ring with adhesive is placed on top of the fibers to create smaller collections of fibers. The leftmost portion of FIG. 9A depicts an uncoated fiber frame (top) and a fiber frame coated with a GelMe film (bottom). FIG. 9B depicts that rings of fibers are dipped into gelatin methacrylate (GelMe) and pulled out at a consistent withdrawal speed to create a thin film around the fiber ring. Then the rings are stacked on top of each other so that the fibers are aligned in a similar direction. FIG. 9C shows confocal imaging of these stacks to observe different characteristics such as thickness and orientation of the nanofibers.

FIG. 10A is from a 10-minute spin time producing a high density of fibers, in accordance with some embodiments. FIG. 10B is of a 5-minute spin time, in accordance with some embodiments. This shorter spin time results in a lower density of fibers.

FIG. 11 depicts dipping aligned nanofibers arrays in a solution of a hydrogel-forming composition to form a thin film, in accordance with some embodiments.

FIG. 12A shows the relationship between the thickness of a single layer film versus the weight percent of GelMe in the dip coating solution, in accordance with some embodiments. FIG. 12B is a 3D image of a 5-layer stack of nanofiber hydrogel, in accordance with some embodiments.

FIGS. 13A-13C are confocal microscope images of dyed nanofibers under different wavelengths of light, in accordance with some embodiments. UV beads that emit a green light were used to dye the nanofibers which allows for better observation of the top of the nanofiber layer. FIG. 13A: Nanofibers dipped in 5% GelMe. FIG. 13B: Nanofibers that have been dipped in 7.5% GelMe. FIG. 13C: Nanofibers dipped in 10% GelMe.

FIGS. 14A-14B are confocal microscope images of the layers within the hydrogel, in accordance with some embodiments. The hydrogel observed had 5 layers of nanofibers within it. All the fibers were aligned and placed such that they all faced the same direction, with the exception of one layer which is placed perpendicular to the others. FIG. 14A: Side view of the hydrogel. FIG. 14B: Top view of the hydrogel stack.

FIG. 15A is Day 1 of seeding the cells onto the middle layer of nanofibers, in accordance with some embodiments. The cells emit a red color while the nanofibers are emitting a blue coloring. FIG. 15B is Day 4 of the experiment. As shown, the cells have attached to the fibers and begun to elongate.

FIGS. 16A-16D demonstrate that the thickness of the nanofiber-hydrogel composite film can be controlled by varying the gelatin percentage, withdrawal speed or temperature, in accordance with some embodiments. FIG. 16A: Thickness vs. gelatin percentage. FIG. 16B: Thickness vs. withdrawal speed. FIG. 16C: Thickness vs. temperature. FIG. 16D: Testing device.

FIGS. 17A-17B demonstrate that the thickness of the nanofiber-hydrogel composite film can be controlled by varying the lateral spacing between NFs and the surface hydrophobicity. in accordance with some embodiments. FIG. 17A: Thickness vs. spin time (which controls the lateral spacing between NFs). FIG. 17B: Thickness vs. PEG percentage (which controls the surface hydrophobicity).

FIGS. 18A-18B demonstrate that the non-limiting example of the nanofiber/hydrogel composites is able to form highly uniform film layers, in accordance with some embodiments.

FIGS. 19A-19B demonstrate that layer mixing is occurring between layers during multilayer structure fabrication, in accordance with some embodiments. A peel test between hydrogel/nanofiber composite layers was performed (FIG. 19A) to prove that layer mixing is occurring between layers during multilayer structure fabrication. The average peeling force is plotted on the right. When a gelatin film at 60° C. is stacked on top of a 22° C. film the peel force required to separate them is more than twice that experience when a gelatin film at 22° C. is stacked on top of another film at 22° C. (FIG. 19B, left two bars). Similarly when a two GelMe films are UV-crosslinked for 3 minutes independently and then stacked the force required to peel them is less then half as large as the force required to peel two films when the first film is UV-crosslinked for 45 s—the second film stacked on the first and then the structure UV-crosslinked for an additional 45 s (FIG. 19B, right two bars).

FIGS. 20A-20B demonstrate that applied forces to the structure during stacking, either due to weight of subsequent stacks or external forces purposefully applied to control layer thickness, affects film thicknesses, in accordance with some embodiments. FIG. 20A: A gelatin-methacrylate structure with several layers that were stacked with the same initial thickness, but were thinner in certain areas of the structure due to applied forces during stacking. FIG. 20B describes this phenomenon associated with the proposed process. Applied force inherent were purposefully controlled during stacking results in thinning of layers between stacks. Composite layers respond differently to the applied force based on extent of temperature and/or UV mediated gelation/crosslinking.

FIGS. 21A-21F demonstrate that cells infiltrate into the multilayer composites, in accordance with some embodiments. FIG. 21A: Schematic of dual-substrate scaffold with axons infiltrating the NF/hydrogel composite region. FIG. 21B: Image of a scaffold with hydrogel control regions surrounding a NF/hydrogel composite. FIG. 21C: Disassembled PDMS holder consists of a rubber ring and two pieces that when assembled form a cylindrical groove to hold a non-limiting 3D NF/hydrogel construct in place. FIG. 21D: Assembled holder supports 3D NF/hydrogel construct up-right for facile cell seeding (with the cell-laden media shown in the middle of the structure). FIG. 21E: Confocal image of cell infiltration inside of the dual-substrate scaffold. Cells form a monolayer on top and migrate only into the graft within the NF region only (do not migrate into control region). FIG. 21F: Migrating cells adopt strong alignment with embedded nanofibers. Red=cell body, blue=NFs and nuclei.

FIG. 22 illustrate a non-limiting example of gradient formation, in accordance with some embodiments. A peptide gradient was formed on the aligned nanofiber array prior to hydrogel embedding. Here norbornene terminated 4-arm poly(ethylene glycol) was mixed at 10% wt/wt with PCL prior to electrospinning to add norbornene groups to the fiber surface. A peptide containing red fluorescent rhodamine and a cysteine group was covalently attached to the fiber surface using a UV light mediated crosslinking procedure. UV exposure time was a gradient using a moving photomask. This resulted in a concentration gradient of peptide cross-linked to the fibers.

### DETAILED DESCRIPTION

Aligned polymer nanofibers promote increased axon alignment and accelerate axon extensions in vitro. For example, aligned nanofibers increased neurite outgrowth and Schwann cell migration by up to 300% when aligned nanofibers were compared to randomly oriented nanofiber controls. Therefore, while not wishing to be limited by theory, in certain embodiments aligned nanofibers can accelerate axon regeneration in vivo and lead to grafts that can guide the healing of nerve gaps greater than 4 cm. Nerve conduits containing nanofiber features do perform better with aligned nanofibers when compared to randomly oriented nanofibers in vivo. However, a major challenge of incorporating aligned nanofibers into an implantable nerve graft is arranging them spatially in a three-dimensional (3D) matrix that is permissive for axon spreading and alignment. Many different strategies have been attempted to incorporate aligned nanofibers into nerve guidance tubes. All previous attempts have utilized dense nanofiber films instead of individual nanofibers due to the difficulty of arranging and handling non-interlaced/bundled nanofibers. Studies with dense nanofiber films include aligned nanofibers on the inner wall of conduit grafts, and variations of aligned nanofiber films folded, rolled, or stacked into various geometries within the conduit core. These approaches have two major limitations: (1) only a small fraction of regenerating axons have direct contact to aligned nanofibers and (2) dense nanofiber films block large portions of the graft, precluding axon regeneration in these regions. Despite these limitations, dense aligned nanofiber film approaches have demonstrated the potential of aligned nanofibers to accelerate axon regeneration and improve functional outcomes.

Therefore, the present disclosure addresses these deficiencies by providing a multi-layer composite with a defined three dimensional structure that can be used as a synthetic tissue graft or as a component of a synthetic tissue graft. In some embodiments, the multi-layer composite comprises two or more sheets of aligned nanofibers coated with a biodegradable hydrogel, wherein the two or more sheets of aligned nanofibers are stacked directly on top of each other and a portion of the biodegradable hydrogel coating on the first sheet is mixed and crosslinked with a portion of the biodegradable hydrogel coating on the second sheet. In some embodiments, the multi-layer composite can be used as a nerve graft or as a component of a nerve graft. In certain embodiments, the multi-layer composite is formed into a tube shape and the outer surface of the tube is coated with an aligned nanofiber outer conduit. In some embodiments, the multi-layer composite coated with the aligned nanofiber out conduit is used as a synthetic nerve graft. In another aspect, the present disclosure provides a method of regenerating an aligned soft tissue in a subject, the method comprising surgically implanting a synthetic tissue graft of the present disclosure in the subject at a site of missing or injured tissue.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, selected methods and materials are described.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” “About” as used herein when referring to a measurable value such as an amount, a temporal duration, and the like, is meant to encompass variations of 20% or ±10%, in certain embodiments ±5%, in certain embodiments ±1%, in certain embodiments ±0.1% from the

As utilized herein, the term “substantially aligned” with regard to the nanofibers of an array includes perfectly aligned, i.e., parallel, fibers as well as fibers that are at a slight angle (e.g., less than about 30°) to one another along all or a portion of their length, such that two fibers may cross one another at some point, but both fibers terminate on collection surfaces spaced apart from one another that are used during automated track electrospinning.

“Aligned nanofiber sheet(s)” and “aligned nanofiber array(s)” are used herein interchangeably.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Compositions

Nanofiber Polymer Composition

In one aspect, the present disclosure relates to a polymer composition that can be used to produce aligned nanofiber sheets using automated track electrospinning. Exemplary polymers that can be used to produce aligned nanofiber sheets include, but are not limited to, polycaprolactone, polylactic acid, alginates, polyacrylonitrile, and combinations thereof. In some embodiments, the polymer used to produce aligned nanofiber sheets is a conductive polymer. Exemplary conductive polymers, include, but are not limited to, poly(3,4-ethylenedioxythiophene), polythiophene, polyaniline, polyacetylene, polyphenylene vinylene, polypyrrole, and combinations thereof. In other embodiments, the polymer used to produce aligned nanofiber sheets is doped such that the polymer is conductive. In certain embodiments, the polymer is doped with carbon nanotubes to form a conductive polymer. In certain embodiments, the polymer used to produce aligned nanofiber sheets is poly(L-lactide) (PLLA). In other embodiments, the polymer used to produce aligned nanofiber sheets is polycaprolactone.

In some embodiments, the polymer used to produce aligned nanofiber sheets is modified such that it comprises a crosslinkable group. In some embodiments, the crosslinkable group is an acrylate, a methacrylate, a thiol, or norbornene. In certain embodiments, a crosslinkable group is incorporated into the aligned nanofiber sheets via automated track electrospinning of a polymer solution, wherein the polymer solution further comprises low molecular weight polymer comprising the crosslinkable group. Although not wishing to be limited by theory, in certain embodiments the low molecular weight polymer can be physically entrapped in the aligned nanofibers formed by electrospinning, thus adding crosslinkable groups to the surface of the aligned nanofibers which make up the electrospun sheets. For example, a solution comprising PLLA and low molecular weight thiolated PLLA (PLLA-SH) can be electrospun via automated track electrospinning to form sheets of aligned PLLA nanofibers comprising crosslinkable thiol groups. In some embodiments, the low molecular weight polymer has a number average molecular weight (Mn) of between about 1000 and about 5000, about 1500 and about 4500, about 2000 and about 4000, about 2000 and about 3000, or about 2250 and about 2750. In some embodiment, the low molecular weight polymer has an Mn of about 2500.

In one embodiment, the polymer composition further comprises a dopant. In some embodiments, the dopant is selected such that it adds a desired biochemical functionality to the aligned nanofiber sheets produced from the polymer composition. In one embodiment, the dopant is collagen. In another embodiment, the dopant is laminin.

In certain embodiments, the polymer composition further comprises a solvent. The solvent can be any solvent known to a person of skill in the art for use in electrospinning nanofibers. Selection of solvent can be of importance in determination of the characteristics of the solution, and hence of the characteristic properties of the nanofibers formed during the electrospinning process. Exemplary solvents include, but are not limited to, acetic acid, acetonitrile, m-cresole, tetrahydrofuran (THF), toluene, dichloromethane (CH2C12), methanol (MeOH), dimethylformamide (DMF), and combinations thereof.

Hydrogel-Forming Composition

In another aspect, the present disclosure relates to a hydrogel-forming composition. In certain embodiments, the hydrogel-forming composition comprises a biodegradable polymer and a crosslinker. Therefore, in some embodiments, the hydrogel-forming composition forms a biodegradable hydrogel.

The biodegradable polymer can be any biodegradable polymer known to a person of skill in the art. In certain embodiments, the biodegradable polymer is gelatin.

In some embodiments, the biodegradable polymer comprises a crosslinkable group. Exemplary crosslinkable groups are described elsewhere herein. In certain embodiments, the crosslinkable group is a methacrylate. In certain embodiments, the biodegradable polymer is gelatin methacrylate (GelMe). In other embodiments, the crosslinkable group is norbornene. In certain embodiments, the biodegradable polymer is gelatin functionalized with norbornene (GelNor). In some embodiments, the gelatin biodegradable polymer is functionalized with norbornene groups via carbodiimide crosslinker chemistry with 1-ethyl-3-(3-dimethylaminopropyl)carbodiimide (EDC) and N-hydroxysuccinimide. This carbodiimide conjugation technique activates the carboxyl groups in the gelatin backbone for direct reaction with 5-norbornene-methylamine via amide bond formation.

In some embodiments wherein the biodegradable polymer comprises a crosslinkable group, the polymer itself may not be biodegradable. However, the addition of biodegradable crosslinkable groups results in a biodegradable polymer. Exemplary polymers which result in a biodegradable polymer of the present disclosure when they comprise a biodegradable crosslinkable group include, but are not limited to, poly(ethylene glycol), hyaluronic acid, collagen, polyacrylamide, and alginate. Exemplary crosslinkable groups are described elsewhere herein.

The crosslinker can be any thermally or light activated crosslinker known to a person of skill in the art. In certain embodiments, the crosslinker is a peptide crosslinker. In some embodiments, the peptide crosslinker is an enzymatically degradable (Deg) peptide comprising thiol end groups. Although not wishing to be limited by theory, it is believed that the bulk properties of a hydrogel formed from the hydrogel-forming composition can be tuned based on the structure of the crosslinker and the amount of crosslinker added to the composition. Therefore, in some embodiments, the bulk hydrogel properties can be tuned by changing the structure and/or concentration of a Deg peptide crosslinker in the composition. In certain embodiments, the Deg peptide has a sequence of GCNSVPMS↓MRGGSNCG (SEQ ID NO:1), wherein the cysteine residues contains thiols that covalently bind to crosslinkable groups on the hydrogel and/or aligned nanofibers, and VPMS↓MRGG (SEQ ID NO:2) is a sequence that cleaves in the presence of cell-secreted matrix metallopeptidases (MMPs). In some embodiments, to tune degradation while keeping total crosslinker peptide concentration constant, non-enzymatically degradable (NonDeg) crosslinker peptides are used in conjunction with Deg peptides. In one embodiment, the NonDeg crosslinker peptide has the sequence GCHGNSGGSGGNEECG (SEQ ID NO:3). In other embodiments, the crosslinker is a non-peptide crosslinker such as dithiothreitol (DTT) or a hyaluronic acid-based macromer comprising a crosslinkable group (e.g. hyaluronic acid modified with norbenene).

In some embodiments, the hydrogel-forming composition further comprises a solvent. In one embodiment, the solvent is an aqueous solvent. In some embodiments, the aqueous solvent is phosphate buffered saline (PBS). In another embodiment, the solvent is an organic solvent which is miscible with water.

In certain embodiments, the hydrogel-forming composition yields a thermoreversible hydrogel. In some embodiments, the hydrogel-forming composition can be cured to form a hydrogel. In certain embodiments, the hydrogel-forming composition can be cured to form a biodegradable hydrogel. In certain embodiments, the hydrogel-forming composition can be cured to form a protease-degradable and/or cell-permissive hydrogel.

Composites

Single Layer Composites

In another aspect, the present disclosure provides a single layer composite comprising an aligned nanofiber sheet coated with a hydrogel. In certain embodiments, the aligned nanofiber sheet comprises a collection of nanofibers that are oriented in a similar direction. In some embodiments, the aligned nanofiber sheet comprises nanofibers with a controlled diameter and spacing. Therefore, in certain embodiments, the composite comprises a sheet of aligned nanofibers coated with a hydrogel such that the composite comprises nanofibers with a specific two dimensional orientation.

The nanofiber sheets are prepared via automated track electrospinning of the nanofiber polymer composition described elsewhere herein. Automated track electrospinning is described in US 2012/0040461, the entire contents of which are incorporated herein by reference. In some embodiments, the density of the aligned nanofibers which form the nanofiber sheets can be controlled by adjusting the collection time.

In one embodiment, the sheets of aligned nanofibers comprise individual nanofibers having a length of between about 0.25 cm to about 100 cm, about 0.25 cm to about 90 cm, about 0.25 cm to about 80 cm, about 0.25 cm to about 70 cm, about 0.25 cm to about 60 cm, about 0.25 cm to about 50 cm, about 0.25 cm to about 40 cm, about 0.25 cm to about 30 cm, about 1.0 cm to about 25 cm, about 1.0 cm to about 20 cm, or about 3.0 cm to about 16 cm. In some embodiments, the individual nanofibers have a length of between about 3.0 cm and about 16 cm.

In some embodiments, the sheets of aligned nanofibers comprise surface modified nanofibers, wherein the modification occurs after automated track electrospinning. In certain embodiments, the surface modification comprises functionalizing the nanofibers with a crosslinkable group. Exemplary crosslinkable groups are provided elsewhere herein. In other embodiments, the surface modification comprises functionalizing the nanofibers with one or more growth factors. In some embodiments, the nanofibers are functionalized with a growth factor gradient along the length of the nanofiber. In certain embodiments, the gradient is a linear increase of about 0 ng/cm³ to about 50 ng/cm³, about 0 ng/cm³ to about 40 ng/cm³, about 0 ng/cm³ to about 30 ng/cm³, about 0 ng/cm³ to about 20 ng/cm³, or about 0 ng/cm³ to about 13 ng/cm³ along the length of the nanofiber. In some embodiments, a growth factor modified with a crosslinkable group is reacted with an aligned nanofiber comprising a crosslinkable group under UV irradiation in order to bind the growth factor to the nanofiber via a crosslinking reaction. In certain embodiments, the nanofibers are functionalized with heparin. In some embodiments, PLLA nanofibers are functionalized with heparin. Therefore, in some embodiments, PLLA nanofibers comprising a crosslinkable thiol are contacted with heparin modified with norbornene under UV irradiation, wherein the thiol and the norbornene react to bind the heparin to the surface of the PLLA nanofibers.

The hydrogel coating is formed from hydrogel-forming composition described elsewhere herein. In one embodiment, the hydrogel coating is a biodegradable hydrogel coating. In certain embodiments, the aligned nanofiber sheet is dipped into a solution comprising the hydrogel-forming composition, coating the aligned nanofiber sheet. In one embodiment, the solution comprising the hydrogel-forming composition comprises an aqueous solvent. In some embodiments, the solvent is PBS. In other embodiments, the solution comprising the hydrogel-forming composition comprises an organic solvent. Although not wishing to be limited by theory, it is believed that the hydrogel coating formed from the hydrogel-forming composition should provide an environment that supports cell survival. Therefore, if the solution comprising the hydrogel-forming composition comprises an organic solvent, an aqueous solvent will then be later infiltrated into the hydrogel coating. In some embodiments, the hydrogel-forming composition is then completely or partially cured to form a hydrogel coating. In some embodiments, the crosslinker in the hydrogel-forming composition functions to covalently bond the aligned nanofibers to the hydrogel matrix. In certain embodiments, when the hydrogel-forming composition is exposed to light or heat, the crosslinkable groups on the aligned nanofibers and/or the hydrogel undergo a reaction with the crosslinker.

In certain embodiments, the single layer composite comprising aligned nanofiber sheets coated with a hydrogel has a thickness of between about 10 m and about 500 m, about 10 m and about 450 m, about 10 m and about 400 m, about 10 m and about 350 m, about 10 m and about 300 m, about 10 m and about 250 m, about 10 m and about 200 m, or about 50 m and about 200 m.

Multi-Layer Compositions

In another aspect, the present disclosure provides a multi-layer composite comprising two or more aligned nanofiber sheets coated with a hydrogel. The aligned nanofiber sheets coated with a hydrogel are described elsewhere herein. In certain embodiments, hydrogel is a biodegradable hydrogel. In certain embodiments, the hydrogel is formed from a crosslinking reaction of the hydrogel-forming composition described elsewhere herein. Therefore, in some embodiments, a biodegradable hydrogel is formed from the crosslinking reaction of a hydrogel-forming composition comprising a biodegradable polymer and a crosslinker. In certain embodiments, the biodegradable hydrogel coating is formed from a crosslinking reaction between crosslinkable groups on the individual aligned nanofibers, crosslinking groups on the biodegradable polymer and the crosslinker. In some embodiments, the hydrogel coating is formed from a crosslinking reaction between GelMe methacrylate groups or GelNor norbornene groups, crosslinkable groups on polycaprolactone or poly(lactic) acid nanofibers, and a crosslinker. Therefore, in some embodiments, the hydrogel coating is covalently bonded to the individual nanofibers which make up the nanofiber sheets. In certain embodiments, the multi-layer composite comprises two or more sheets of substantially aligned polycaprolactone or poly(lactic) acid nanofibers coated with a gelatin hydrogel.

In certain embodiments, the multi-layer composite has a defined three dimensional structure due to the controlled diameter and spacing of the nanofibers in the aligned nanofiber sheets as well as defined spacing between the two or more aligned nanofiber sheets coated with a hydrogel that form the multi-layer composite. In certain embodiments, the spacing between the two or more aligned nanofiber sheets coated with a hydrogel is controlled by the thickness of the single layer composite.

In some embodiments, the multi-layer composite comprises two or more sheets of aligned nanofibers coated with a biodegradable hydrogel, wherein the two or more sheets of aligned nanofibers are stacked directly on top of each other and a portion of the biodegradable hydrogel coating on the first sheet is mixed and crosslinked with a portion of the biodegradable hydrogel coating on the second sheet. In some embodiments, the two or more sheets of aligned nanofibers are stacked directly on top of each other such that the second sheet completely covers the first sheet. In certain embodiments, when the multi-layer composite comprises more than two sheets of aligned nanofibers, the additional sheets are stacked on top of the first or second sheet such that the additional sheets completely cover the first or second sheet. In some embodiments the two or more sheets of aligned nanofibers are stacked such that the individual aligned nanofibers in each sheet are substantially aligned in the same (two dimensional) direction.

In certain embodiments, the biodegradable coating on the first sheet has mixed with the biodegradable hydrogel coating on the second sheet during the fabrication of the multi-layer composite. In some embodiments, the multi-layer composite is fabricated using a method described elsewhere herein. In certain embodiments, the mixing of a portion of the hydrogel coating on the first sheet with a portion of the hydrogel coating on the second sheet yields a cohesive multi-layer composite with interdigitation between adjacent hydrogel coatings and a defined three dimensional structure.

In some embodiments wherein the single layer composite is formed by dipping the aligned nanofiber sheet into a solution comprising an organic solvent and the hydrogel-forming composition, the multi-layer composite formed from two or more sheets of the single layer composite is later infiltrated with an aqueous solvent which replaces any remaining organic solvent. In one embodiment, the multi-layer composite is infiltrated with an aqueous solvent by immersing the multi-layer composite in the aqueous solvent. In some embodiments, the aqueous solvent is PBS.

Methods

Method of Preparing a Multi-Layer Composite

In another aspect, the present disclosure relates to a method of preparing a multi-layer composite with a defined three dimensional structure, the method comprising: (a) electrospinning a polymer composition to form a sheet of aligned nanofibers; (b) coating the sheet of aligned nanofibers with a thermoreversible hydrogel-forming composition; (c) cooling a first coated aligned nanofiber sheet such that the thermoreversible hydrogel-forming coating forms a gel; (d) stacking a second coated aligned nanofiber sheet on top of the first coated aligned nanofiber sheet to form a multi-layer composite, wherein the second coated aligned nanofiber sheet is maintained at a temperature such that the thermoreversible hydrogel-forming coating is in a liquid state; (e) optionally stacking one or more coated aligned nanofiber sheets on the multi-layer composite such that the additional coated aligned nanofiber sheets that are cooled are adjacent to coated aligned nanofiber sheets that are at an elevated temperature; and (f) curing the multi-layer composite.

In step (a), the aligned nanofiber sheet is formed by automated track electrospinning of a nanofiber polymer composition described elsewhere herein. In certain embodiments, the aligned nanofiber sheet comprises individual nanofibers having a length of between about 0.25 cm to about 100 cm, about 0.25 cm to about 90 cm, about 0.25 cm to about 80 cm, about 0.25 cm to about 70 cm, about 0.25 cm to about 60 cm, about 0.25 cm to about 50 cm, about 0.25 cm to about 40 cm, about 0.25 cm to about 30 cm, about 1.0 cm to about 25 cm, about 1.0 cm to about 20 cm, or about 3.0 cm to about 16 cm. In some embodiments, the individual nanofibers have a length of between about 3.0 cm and about 16 cm.

In certain embodiments, the aligned nanofiber sheet comprises GelMe nanofibers that are substantially aligned with one another. In certain embodiments, the aligned nanofiber sheet comprises GelNor nanofibers that are substantially aligned with one another.

In some embodiments, step (b) is preceded by step (a′), comprising modifying the surface of the nanofibers which make up the sheets of aligned nanofibers. Exemplary surface modifications are described elsewhere herein.

In step (b), the thermoreversible hydrogel-forming composition is described elsewhere herein as the hydrogel-forming composition. Therefore, the thermoreversible hydrogel-forming composition comprises a biodegradable polymer and a crosslinker. In certain embodiments, the thermoreversible hydrogel-forming composition comprises a crosslinker and gelatin that has been modified with a crosslinkable group. In some embodiments, the thermoreversible hydrogel-forming composition comprises a crosslinker and GelMe or GelNor. The aligned nanofiber sheet can be coated with a thermoreversible hydrogel-forming composition using any method known to a person of skill in the art. In some embodiments, the nanofiber sheet is coated with the thermoreversible hydrogel-forming composition by dipping the nanofiber sheet in a solution comprising the thermoreversible hydrogel-forming composition wherein the solution is maintained at an elevated temperature such that the biodegradable polymer is in a liquid state. In certain embodiments, the thickness of the coating is controlled by varying the concentration of the biodegradable polymer in the dipping solution.

In step (c), the first coated aligned nanofiber sheet can be cooled to any temperature needed in order for the thermoreversible hydrogel-forming coating to form a gel. In some embodiments wherein the thermoreversible hydrogel-forming coating comprises gelatin or gelatin modified with a crosslinkable group, the first coated aligned nanofiber sheet is cooled to a temperature of between about 17° C. and 22° C. In certain embodiments wherein the thermoreversible hydrogel-forming coating comprises gelatin or gelatin modified with a crosslinkable group, the first coated aligned nanofiber sheet is cooled to a temperature of about 20° C. In some embodiments, the lower temperature causes partial, reversible, curing of the thermoreversible hydrogel-forming coating to form a gel, without permanently curing the coating to form a hydrogel. Therefore, in some embodiments, the lower temperature creates thermoreversible bonds in the coating which will break at elevated temperatures.

In step (d), the second coated aligned nanofiber sheet is stacked directly on top of the first coated aligned nanofiber sheet. In certain embodiments, the second sheet is stacked on top of the first sheet such that the aligned nanofibers making up both sheets are arranged in the same two dimensional orientation. In certain embodiments, stacking the second sheet wherein the thermoreversible hydrogel-forming coating is in a liquid state on top of the first sheet causes local melting of the cooled coating on the first sheet. In some embodiments, this local melting leads to continuous fusion and/or interdigitation between the coating of the first sheet and the coating of the second sheet.

In step (e) additional coated aligned nanofiber sheets may be added to the multi-layer composite formed from the first and second coated aligned nanofiber sheets. Additional coated aligned nanofiber sheets that have been cooled to form a gel coating are stacked such that they are directly adjacent to aligned nanofiber sheets wherein the coating is in a liquid state. In certain embodiments, the additional coated aligned nanofiber sheets are added to the multi-layer composite such that the aligned nanofibers making up the additional sheet are arranged in the same two dimensional orientation as the sheets which make up the multi-layer composite.

In step (f), the multi-layer composite can be cured using any method known to a person of skill in the art. In some embodiments, the multi-layer composite is cooled before curing such that some or all of the thermoreversible hydrogel-forming coating that is in a liquid state forms a gel. In certain embodiments, the multi-layer composite is cured by irradiating the composite with UV light. In certain embodiments, curing the multi-layer composite causes a crosslinking reaction between the crosslinker and the biodegradable polymer in the thermoreversible hydrogel-forming coating, forming a hydrogel. In some embodiments wherein the aligned nanofibers comprise a crosslinkable group, curing the multi-layer composite causes a crosslinking reaction between the crosslinker and the aligned nanofibers. In other embodiments wherein the aligned nanofibers comprise a crosslinkable group, curing the multi-layer composite causes a crosslinking reaction between the crosslinker, the biodegradable polymer, and the aligned nanofibers. In certain embodiments, due to the continuous fusion and/or interdigitation of the coating between adjacent aligned nanofiber sheets, curing the multi-layer composite causes crosslinks between the coatings of the adjacent nanofiber sheets.

Method of Regenerating Aligned Soft Tissues

In yet another aspect, the present disclosure relates to a method of regenerating an aligned soft tissue in a subject, the method comprising surgically implanting a synthetic tissue graft in the subject at a site of missing or injured tissue, wherein the synthetic tissue graft is a multi-layer composite comprising two or more sheets of aligned nanofibers coated with a biodegradable hydrogel wherein the two or more sheets of aligned nanofibers are stacked directly on top of each other and a portion of the biodegradable hydrogel coating on the first sheet is mixed and crosslinked with a portion of the biodegradable hydrogel coating on the second sheet.

The aligned soft tissue can be any aligned soft tissue in the subject. Exemplary aligned soft tissues include, but are not limited to, nerve, spinal cord, skeletal muscle, smooth muscle, cardiac muscle, and combinations thereof. In certain embodiments, the aligned soft tissue is a nerve. Therefore, in certain embodiments, the synthetic tissue graft is a nerve graft which bridges a missing or injured part of a nerve.

The multi-layer composite is described elsewhere herein. In some embodiments, each of the two or more sheets of aligned nanofibers comprise individual nanofibers having a length of between about 0.25 cm to about 100 cm, about 0.25 cm to about 90 cm, about 0.25 cm to about 80 cm, about 0.25 cm to about 70 cm, about 0.25 cm to about 60 cm, about 0.25 cm to about 50 cm, about 0.25 cm to about 40 cm, about 0.25 cm to about 30 cm, about 1.0 cm to about 25 cm, about 1.0 cm to about 20 cm, or about 3.0 cm to about 16 cm. In some embodiments, the individual nanofibers have a length of between about 3.0 cm and about 16 cm.

In certain embodiments, each of the two or more sheets of aligned nanofibers are individually completely encapsulated in a biodegradable hydrogel coating such that the hydrogel coating covers the entire sheet of aligned nanofibers. The biodegradable hydrogel coating is formed from the hydrogel-forming composition described elsewhere herein. Therefore, the biodegradable hydrogel coating is formed from a crosslinking reaction between a crosslinker and a biodegradable polymer comprising a crosslinkable group. In some embodiments, the biodegradable polymer comprising a crosslinkable group is GelMe or GelNor. In some embodiments, the multi-layer composite comprises more than two sheets. In embodiments wherein the multi-layer composite comprises more than two sheets, the additional sheets are stacked directly on top of the first or second sheet and a portion of the biodegradable hydrogel coating on the additional sheet(s) is crosslinked with a portion of the biodegradable hydrogel coating on the sheet(s) directly adjacent to the additional sheet(s).

In some embodiments, the multi-layer composite is constructed as described elsewhere herein. Therefore, in some embodiments, sheets of aligned nanofibers coated with a gelled thermoreversible hydrogel-forming coating are stacked directly adjacent to sheets of aligned nanofibers coated with a liquid thermoreversible hydrogel-forming coating. In certain embodiments, stacking sheets with a gelled coating directly adjacent to sheets with a liquid coating causes local melting of the gelled coating and mixing between the locally melted gelled coating and liquid coating. Curing of the multi-layer composite with mixing between the coating of adjacent sheets forms the hydrogel coating with a portion of the biodegradable hydrogel coating on the first sheet crosslinked with a portion of the biodegradable hydrogel coating on the second sheet.

In certain embodiments, the multi-layer composite comprises a defined three dimensional structure. In some embodiments, the two dimensional structure of the multi-layer composite is defined by the controlled diameter and spacing of the individual nanofibers which make up the sheets of aligned nanofibers. In some embodiments, the three dimensional structure of the multi-layer composite is defined by stacking sheets of aligned nanofibers with controlled diameter and spacing which are coated with a biodegradable hydrogel coating, wherein the coated sheet has a known and/or controlled thickness.

In some embodiments wherein the method is used to regenerate a nerve in the subject, the synthetic tissue graft further comprises an outer conduit. In certain embodiments, the synthetic tissue graft is formed into a rod or cylinder shape wherein the outer conduit is on the outside of the rod or cylinder. In one embodiment, rod or cylinder of synthetic tissue graft is inserted into an outer conduit that is in the shape of a hollow tube. In certain embodiments, the outer conduit results in increased structural stability and forms a readily implantable graft with strong mechanical attachment between the outer conduit and inner hydrogel composite. In one embodiment, the outer conduit is a porous, solid, tube. In another embodiment, the outer conduit is an aligned nanofiber outer conduit. In some embodiments, the outer conduit comprises circumferentially aligned polycaprolactone nanofibers.

Although not wishing to be limited by theory, it is believed that aligned nanofibers in the synthetic tissue graft promote cell growth and alignment compared to randomly oriented nanofibers. It is also believed that the defined three dimensional structure of the disclosed synthetic tissue graft cures the deficiencies found in conventional synthetic tissue grafts which comprise dense films of aligned nanofibers lacking a defined three dimensional structure. Specifically, it is believed that the disclosed synthetic tissue graft addresses the following problems found in conventional grafts: (1) only a small fraction of regenerating cells have direct contact to aligned nanofibers and (2) dense nanofiber films block large portions of the graft, precluding cell regeneration in these regions. In certain embodiments, the biodegradable hydrogel coating is enzymatically degradable, protease-degradable, and cell permissive. Therefore, in some embodiments, the biodegradable hydrogel permits cell infiltration and allows the cells to grow into the synthetic tissue graft while the nanofibers provide spatial support. Although not wishing to be limited by theory, it is believed that as the regenerating cells locally remodel the hydrogel via metallopeptidase (MMP) mediated degradation, they sense mechanical cues from the aligned nanofibers allowing for directional cellular infiltration while maintaining continued support for the aligned nanofiber architecture.

In some embodiments wherein the method is used to regenerate a nerve in the subject, the synthetic tissue graft comprises sheets of aligned nanofibers wherein the nanofibers are longer than about 3 cm. In some embodiments, the nanofibers are between about 3 cm and about 16 cm in length. Therefore, in certain embodiments, the disclosed synthetic tissue graft comprising aligned nanofibers that are longer than about 3 cm can be used to heal and/or regenerate a nerve gap in the subject that is longer than about 3 cm. It is thus believed that the disclosed tissue grafts can be used to treat nerve injuries and nerve gaps that span longer distances when compared to conventional tissue grafts, which treat nerve injuries and nerve gaps of about 1 cm to about 3 cm.

Synthetic Tissue Graft

In yet another aspect, the present disclosure relates to a synthetic tissue graft. In certain embodiments, the synthetic tissue graft promotes the healing and/or regeneration of an aligned soft tissue in a subject. Exemplary aligned soft tissues are described elsewhere herein. The synthetic tissue graft is described elsewhere herein. Therefore, the synthetic tissue graft comprises the multi-layer composite described elsewhere herein. In some embodiments, the synthetic tissue graft is a synthetic nerve graft which is used to promote healing and/or regeneration of a nerve injury or nerve gap. In some embodiments wherein the synthetic tissue graft is a synthetic nerve graft, the synthetic nerve graft further comprises an outer conduit covering the outside surface of the nerve graft. The outer conduit is described elsewhere herein. In certain embodiments, the synthetic nerve graft promotes healing and/or regeneration of a nerve injury or nerve gap that is longer than about 3 cm.

Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Aligned Nanofibers Spatially Arranged in a 3-Dimensional Matrix

Materials and Methods

### Nanofiber Array Fabrication

Nanofiber-hydrogel composites were fabricated using a two-step process. First, aligned nanofiber arrays with controlled nanofiber diameter and spacing were electrospun from poly(L-lactide) (PLLA) (Lactel Absorbable Polymers, 1.09 dL/g inherent viscosity) solutions dissolved in chloroform at 10% wt/v. Low-molecular weight thiolated PLLA (PLLA-SH, Mn-2500, Sigma) was added to PLLA solutions at 2% wt/v. The low molecular weight PLLA-SH molecules in the PLLA solution are physically entrapped in the resultant blended nanofibers, adding thiol groups to the surface of PLLA nanofibers without the need for subsequent modifications. A similar one step blending-based surface modification approach was previously developed to attach heparin to nanofiber surfaces. The available thiol groups serve to (1) covalently crosslink nanofibers to the GelNor hydrogel matrix to stabilize architecture and (2) functionalize nanofibers with growth factors gradients via heparin binding. It has been shown that covalent binding of randomly oriented nanofibers to a hydrogel matrix can enhance nanofiber bioactivity.

### Ge/Nor Hydrogels

Gelatin was functionalized with norbornene groups via carbodiimide crosslinker chemistry with 1-ethyl-3-(3-dimethylaminopropyl)carbodiimide (EDC) and N-hydroxysuccinimide. This carbodiimide conjugation technique activates the carboxyl groups in the gelatin backbone for direct reaction with 5-norbornene-methylamine via amide bond formation.

GelNor macromers can be crosslinked with enzymatically degradable (Deg) peptides with thiol end groups by light-mediated reactions. Thus, bulk hydrogel degradation properties can be tuned based on the structures and quantities of the Deg peptides selected. It was recently shown that cells encapsulated in hydrogels with Deg crosslinker peptides (sequence: GCNSVPMS↓MRGGSNCG) spread to varying degrees depending on Deg concentration. The cysteine “C” residue contains thiols that covalently bind to norbornenes in GelNor, and VPMS↓MRGG is a sequence that cleaves in the presence of cell-secreted matrix metallopeptidases (MMP). To tune degradation while keeping total crosslinker peptide concentration constant, non-enzymatically degradable (NonDeg) crosslinker peptides (sequence: GCHGNSGGSGGNEECG) will be used alongside Deg crosslinker peptides.

Functionalizing Individual Nanofibers with NGF Gradients

To tether growth factors onto nanofibers, nanofibers will undergo heparin surface modification. PLLA-SH nanofibers will be incubated in Heparin-Nor and exposed to UV light to bind heparin to surface thiols via a thiol-norbornene reaction. Heparin gradients will be induced along the length of nanofibers via a sliding mask approach. A similar approach has been used previously to create peptide gradients in hydrogels. Next, heparin-bound PLLA nanofiber arrays will be incubated in NGF to allow for binding to heparin using a previously described procedure to obtain a desired surface density of ˜13 ng/cm² bound NGF. The targeted gradient will be a linear increase from 0 to ˜13 ng/cm² along the longitudinal direction. NGF binding will be confirmed via immunofluorescence-based analysis and ELISA.

### Hydrogel Layer Thickness

Layer thickness is a critical parameter required to control fiber density because the distance between fibers in the stacking direction is equal to the layer thickness. Several dip coating parameters affect film thickness, including withdrawal speed and solution viscosity, which is dependent on gelatin concentration and dipping temperature. Experiments will be performed to determine the upper and lower limits and repeatability of layer thickness for films made with 2.5, 5, and 10% wt/v Gel-Nor-nanofiber films at different withdrawal speeds and dipping temperatures.

### Investigation of the Effect of Layer Thickness on Neural Cell Invasion

Multilayer constructs will be made 1 cm long×2 mm thick then wrapped with a conduit. They will be cut into 2 mm sections and primary neurons will be seeded on the cross-section. Fiber diameter spacing in each layer will be determined based on findings that maximized cell elongation and alignment. Layer thicknesses will be modified to include low, medium, and high thickness groups.

Construction of Aligned Nanofiber-Hydrogel Composite Nerve Conduits with Clinically Relevant Geometries

Grafts will be constructed with a 2.5 mm diameter (similar to that of the ulnar nerve proximal to the elbow) and 10 cm in length (a length that well exceeds those currently used in common clinical nerve grafting procedures). Currently marketed nerve grafts do not demonstrate sufficient regeneration at distances>3 cm, so length is limited by performance. However, grafts up to 10 cm in length may be of clinical interest.

### Evaluation of Long-Term Stability of Nanofiber-Hydrogel Synthetic Grafts

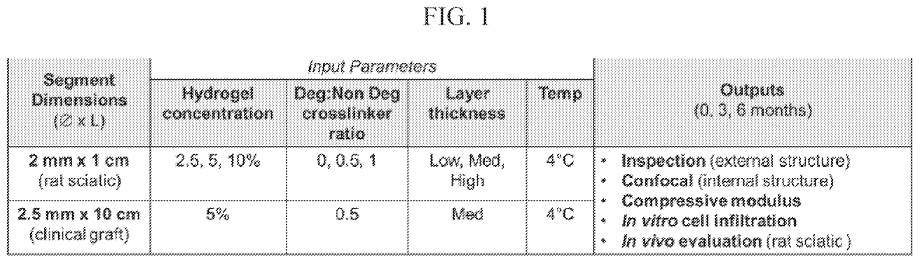

Long-term stability of the constructs are evaluated (gross geometry, internal nanofiber architecture, mechanics, and bioactivity) for up to six months, to evaluate the utility of this graft as an off-the-shelf product. Grafts with input parameters summarized in FIG. 1 and geometries for the rat sciatic nerve and clinical applications are constructed under sterile conditions and cut into 2 mm long segments. Segments are incubated at 4° C. under sterile conditions for κ, 3, or 6 months. Samples are visually inspected for any extrusion of the graft core from the conduit and the outer dimensions of the graft will be measured. Some segments are imaged fluorescently to visualize the dyed internal nanofiber architecture (distance between layers, nanofiber alignment and density in the longitudinal and frontal planes). Other segments are subject to compression testing of the nanofiber-hydrogel core. Remaining segments are seeded with primary neurons on their cross-sectional surface. Cells are fixed at 1, 3 & 7 days and cell penetration is measured quantitatively based on the average depth of cell penetration into the structure.

### Multilayer Structure Fabrication

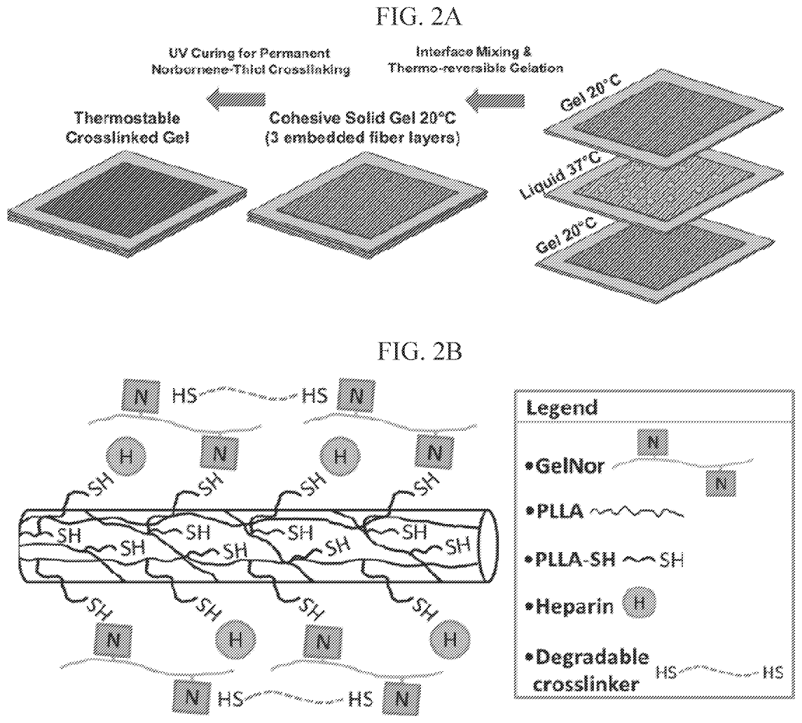

Hydrogels formed with GelNor macromers are ideal for constructing multilayered nanofiber-hydrogel structures due to thermo-reversible and photocrosslinkable properties GelNor in solution is liquid above 37° C., forms a semi-stable hydrogel at room temperature, and will go back into solution as temperature is increased. To leverage this thermo-reversible phenomenon, a nanofiber-hydrogel composite layer was formed by dipping a nanofiber sheet into hydrogel solution (GelNor macromer & crosslinker peptide) at an elevated temperature (e.g., 37° C.) and allowed to self-form into a soft nanofiber-hydrogel composite film as it cooled to room temperature (20° C.). Nerve regeneration in vivo occurs in 3D, so cellular interactions need to be evaluated within a multilayer cohesive 3D structure. Thus hydrogel liquid solution layers at an elevated temperature were stacked in contact with gelled hydrogel composite layers (20° C.) and cooled to room temperature (FIG. 2A). Thermo-reversible gel (20° C.) and liquid layers (37° C.) were alternated so that local melting at the interface between layers results in good bonding and a seamless 3D solid structure. Once the stack was complete, the nanofiber-hydrogel composite was irradiated with UV light (5 mW/cm², 5 min) to form covalent crosslinks between norbornene in GelNor macromers and thiols on crosslinker peptides and exposed PLLA-SH on the nanofibers surface (FIG. 2B).

### Cell Culture (Nanofiber Hydrogel Parameters)

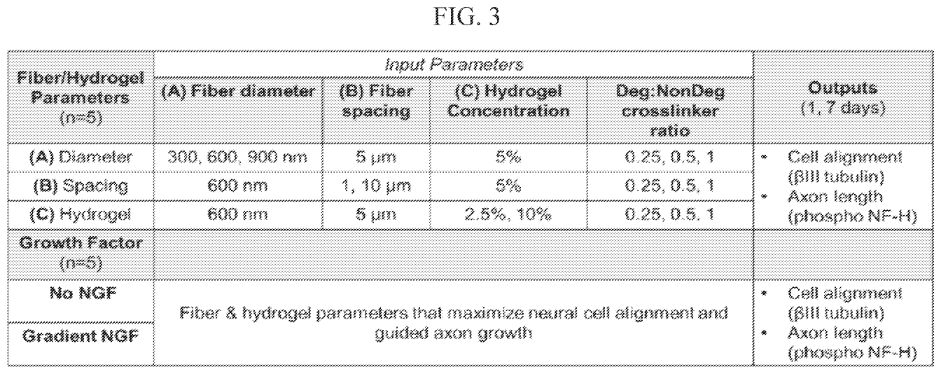

Primary rat neurons (1×10⁶ cells/ml, Lonza) are encapsulated in the center layer of nanofiber-hydrogel composites (3 layers thick) and used to evaluate the effects of nanofiber (diameter, spacing) and hydrogel (GelNor concentration, Deg:NonDeg ratio) parameters on axon elongation and alignment. The input variables are nanofiber diameter (300, 600, 900 nm), nanofiber spacing (1, 5, 10 μm gap per fiber), gelatin solution concentration (2.5, 5, 10%) and crosslinker peptide ratios between Deg and Non-Deg peptides (0.25, 0.5, 1 Deg:NonDeg ratio), resulting in a total of 21 groups and 105 samples (n=5) per time-point (FIG. 3). The initial nanofiber parameters are based on published work on the effects of nanofibers on axon growth, and the Deg crosslinker peptides modulate cell spreading in 3D hydrogels in a concentration-dependent manner. Each group contains five replicates (n=5) with outputs of neural cell alignment with respect to nanofiber direction and axon length which are measured using confocal images of neural cells stained for phosphorylated neurofilament (phospho NF—H). Cellular outputs are evaluated after 1- and 7-days in culture for all groups.

### Cell Culture (Growth Factor Parameters)

Fiber diameter, spacing, GelNor concentration, and Deg:NonDeg crosslinker ratio that maximizes neural cell alignment and guided axon growth will be used as design parameters for a second in vitro study which will investigate the effect of NGF gradients on enhanced neural cell outcomes. Constructs will contain a longitudinal NGF gradient (0-13 ng/cm²) in the direction of the aligned nanofibers (FIG. 3). Cell alignment and axon length will be quantified after 1- and 7-days to evaluate potential additive effects from NGF gradients.

### Rat Sciatic Nerve Gap Model

To transect the sciatic nerve, a dorsolateral gluteal muscle-splitting incision of 2-month-old mixed male and female Lewis rats will expose the sciatic nerve which will be transected (1 cm gap) with a straight microscissor approximately 1 cm distal to the sciatic notch. The proximal and distal nerve stumps will then be bridged with the different implant groups, which will be separated into two studies. All in vivo studies will include equal proportions of male and female animals for each experimental group. Experimental groups will be randomly assigned within each gender of rats. The first study (Degradation Study, DS) will focus on investigating how the in vivo microenvironment affects nanofiber-hydrogel graft degradation kinetics and performance. Nanofiber-hydrogel composite grafts (n=8 for each group) will be tested according to 3 groups (24 rats in total) as follows: (DS1) constructs fabricated with hydrogel and nanofiber properties found to induce maximal axon elongation in vitro (optimal gel concentration, Deg:NonDeg crosslinker ratio, nanofiber diameter, lateral spacing, and layer thickness), (DS2) same construct parameters as (DS1) but with half of the Deg crosslinker ratio to delay increased enzymatic degradation that may occur faster in an in vivo microenvironment, and (DS3) constructs with hydrogel parameters to minimize in vivo degradation. DS3 parameters will be fixed at 600 nm nanofiber diameter, 5 μm lateral nanofiber spacing, 25 μm thickness per layer, and with a high GelNor concentration (10 wt %) without any Deg crosslinker (Deg:NonDeg crosslinker ratio of 0).

To evaluate gait and electrophysiology, the sciatic functional index (SFI) and compound muscle action potential (CMAP) recordings will be performed at four-week intervals (4, 8, 12 weeks). The SFI is a widely used metric that is determined by comparing the geometric representation of the affected hind paw print to the contralateral paw print during walking. Scoring ranges from 10 (normal) to 100 (injured). Electrophysiology will be performed on rats under isoflurane anesthesia by placing stimulating electrodes at the sciatic nerve (proximal and distal to the graft) and recording the CMAP electrode over the gastrocnemius muscle. CMAP peak amplitude and latency will be recorded and nerve conduction velocity will be calculated based on distance between the stimulating electrodes.

At twelve weeks post-surgery, the rats will be euthanized. The twelve-week timepoint has been found to be enough time for axon growth and improvements in functional outcomes and is the most common time-point used in rat sciatic nerve studies. Shortly post-euthanasia, rats will be perfused with 3% glutaraldehyde or 4% paraformaldehyde for resin or paraffin embedding respectively. Nerve grafts will be harvested, assessed for graft integrity, or signs of infection and post-fixed in 3% glutaraldehyde then 1% osmium tetroxide or 4% paraformaldehyde prior to resin or paraffin embedding, respectively. Equivalent numbers of samples will be embedded in resin and paraffin. Resin embedded samples will be cut into 1 μm cross-sections and stained with toluidine blue. Slides will be imaged with light microscopy to quantify myelinated axon count and diameter at 2, 5, and 8 mm from the proximal suture. Samples embedded in paraffin will be sectioned longitudinally and stained with H&E to examine general morphology and lymphocytic infiltration and immunostained with primary antibodies against the following: 3III tubulin and phospho NF—H (to visualize axons and measure axon elongation distance into the graft), myelin basic protein (MBP, to visualize myelinated axon length), S-100 (to visualize Schwann cell infiltration), and ED128 and F4/8067 (to visualize macrophages as an indicator of inflammation). Increased inflammatory cell number has been correlated with improved SFI outcomes in wild type mice versus mice with downregulated innate immune response after sciatic nerve injury. The gastrocnemius muscles will be excised from the operated and non-operated hind limb and the ratio of their wet weight will be measured as an assessment of muscle degeneration associated with denervation. Muscles will be embedded in paraffin, sectioned and stained with H&E and Masson's trichrome. Muscle cell cross-sectional area will be quantitatively measured, and fibrous connective tissue deposition will be qualitatively assessed. All histologic analysis will be performed by a blinded pathologist to minimize bias in assessment.

The graft design found to have the best SFI functional outcome in the Degradation Study will be used to define nanofiber and hydrogel parameters tested in a second more elaborate animal study (Full Study, FS). If no group is statistically superior in SFI score, then a statistically superior group for nerve conduction velocity or myelinated axon count at center graft may be used instead of SFI. In this study, 5 groups (n=20 for each group) will be explored as follows: (FS1) best nanofiber-hydrogel parameters identified in the Degradation Study (DS*), (FS2) same as FS1, but after graft sterilized in 1N HCl and stored in sterile PBS at 4° C. for 6 months, (FS3) same as FS1, but with a NGF gradient (0-13 ng/cm² from proximal to distal end), (FS4) allografts from donor rats (positive control), and (FS5) hollow nerve grafts (outer conduit only, negative control). Each donor rat provides two nerves so 110 rats are required (20 rats/group+10 rats for allograft group). Surgeries, functional testing, and histology will be performed as described above.

### Power Analysis and Statistics

To estimate an effect size similar to the allograft versus conduit control group, a power analysis using the means and standard deviation (σ) for autograft and random nanofiber hollow conduit in a similar published study was performed. Alpha was set to 0.01 and power was set to 0.9. Sciatic index means were 55 and 61 with σ=4. Axon count means were 13,000 and 11,500 with σ=700. Analysis resulted in n=14 for sciatic index and n=7 for mid-graft axon count. Other reports of similar studies reported loss of ˜20% of animals to autotomy, wounds, etc. Thus n=20 for behavioral testing and n=10 for histological testing, as proposed, provide appropriate sample size accounting for potential animal or sample loss. FS power analysis may be adjusted after analyzing data from the Degradation Study. For all experiments, statistical difference between two or more groups will be determined by Kruskal-Wallis (p<0.05) and subsequent group by group comparisons will be conducted with Mann-Whitney (p<0.05) tests.

Results and Discussion

The current inability to engineer a nerve conduit with well-distributed individual aligned nanofibers is due to an inadequate matrix for holding nanofibers in place and poor control over assembling nanofiber architectures (nanofiber orientation in three-dimensions and nanofiber packing density). To address these limitations, the present disclosure uses a layer-by-layer assembly approach that embeds aligned nanofiber arrays into cell permissive hydrogels. Gelatin modified with crosslinkable groups was used herein as the cell permissive hydrogel. Gelatin is an enzymatically degradable and temperature responsive polymer that can be modified with functional groups, allows for the encapsulation of cells and nanofibers, and offers a wide range of biophysical properties. Specifically, gelatin modified with norbornene groups (GelNor) allows for light-controlled click reactions between norbornene and thiol-containing crosslinker peptides to form hydrogels. The present Inventor have pioneered technologies for producing long, low-density polymer nanofiber arrays with highly controlled packing density, and nanofiber alignment. These arrays contain continuous individual nanofibers (e.g., Poly(L-lactic acid), PLLA) with clinically relevant lengths (up to tens of centimeters). Neural cells extend aligned neurites when cultured directly on these arrays. In certain embodiments, axon growth/regeneration can be accelerated in composite hydrogel grafts by arranging aligned nanofibers within the supportive hydrogel matrix in precise, stable 3D architectures.

Certain following non-limiting innovations were used to prepare the stable 3D architectures of the present disclosure.

Automated track electrospinning. This technique uses a parallel automated track collecting device to precisely assemble polymer nanofiber structures. The technique can produce ultra-thin aligned nanofiber sheets with individual fiber lengths up to 16 cm and unlimited width. Nanofiber density (lateral spacing between nanofibers) can also be tightly controlled. Single-layer nanofiber sheets with these specifications cannot be easily produced by any other method. Neural cells extended aligned axons when cultured on these substrates.

Layer-by-layer aligned nanofiber-hydrogel matrix composite assembly. Recently developed methods were used to fabricate 3D structures by stacking multiple single-layer nanofiber sheets embedded in permissive hydrogels. A dip coating method was used to control hydrogel thickness within each layer. Thermoreversible changes between hydrogel solid/liquid phases and photopolymerization reactions to covalently crosslink composites were leveraged to adhere multiple single-layer nanofiber sheets into a cohesive 3D structure. Nanofiber density control in the lateral direction (via automated track electrospinning) and layer thickness control in the vertical direction (via dip coating parameters) allowed for the precise tuning of nanofiber packing density throughout the scaffold.

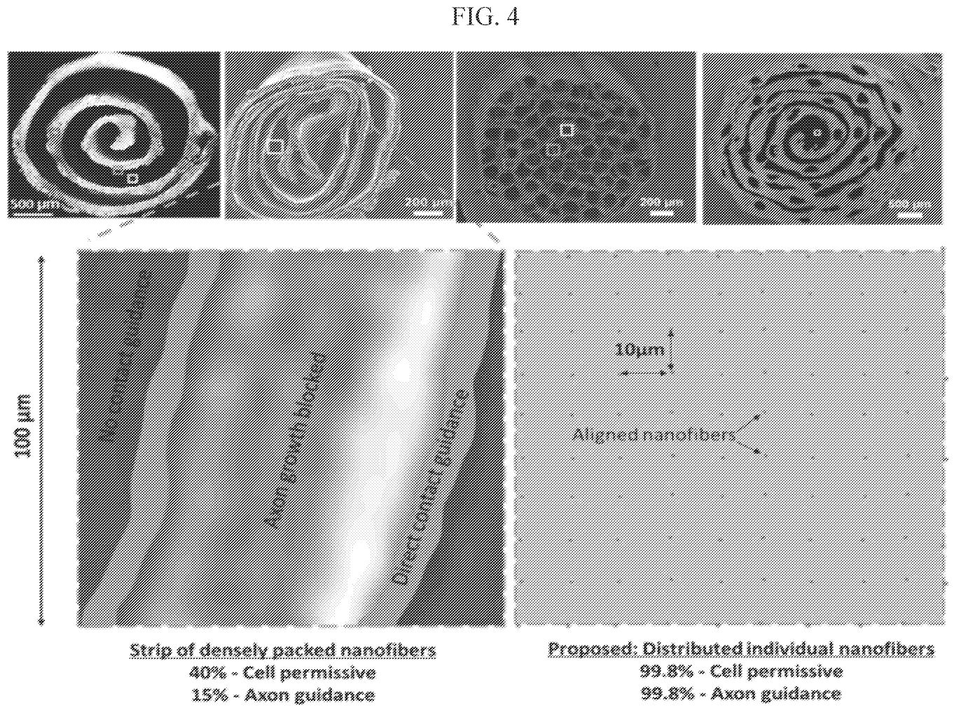

Hydrogel matrix support allows for existence of individual nanofibers. Aligned nanofibers are too delicate to exist individually in a hydrated three-dimensional space without any supporting structure. Thus, previous attempts to incorporate aligned nanofibers into tissue engineering grafts have utilized densely packed films of nanofibers or placed nanofibers on a larger structure, such as the inner wall of a tubular conduit, for support. Dense fiber films block axon growth (FIG. 4, red squares) through large sections of the graft and may hinder lateral cell-cell communication. Here, individual nanofibers were supported by a hydrogel matrix to stabilize nanofiber architecture in three-dimensional space, allowing for lateral spacing control and the existence of individual nanofibers. Furthermore, nanofibers were covalently crosslinked to the hydrogel matrix to ensure stability of the desired internal nanofiber architectures.

Hydrogel-aligned nanofiber composite permissive to cellular infiltration. In nanofiber-based tissue engineering grafts, dense nanofiber mats typically prevent cell infiltration (FIG. 4). Here, a protease-degradable hydrogel matrix allows cells to grow into the composite while providing spatial nanofiber support. As cells locally remodel the hydrogel via metallopeptidase (MMP) mediated degradation, they sense mechanical cues from aligned nanofibers allowing for directional cellular infiltration while maintaining continued support for the aligned nanofiber architecture.

Peripheral nerve graft with evenly distributed continuous aligned individual nanofibers. No graft has ever been constructed that contains individual, continuous (up to 16 cm end to end) aligned nanofibers with spacing control. Nanofiber spacing control is a major breakthrough since it allows for tunable axon-nanofiber interactions providing increased direct contact between axons and nanofibers without dense fiber mats that block paths for axon growth.

Identify Nanofiber-Hydrogel Composite Parameters that Maximize Neural Cell Alignment and Axon Growth In Vitro

Based on previous studies, physical cues can guide axon alignment and accelerate elongation in 2D and 3D systems. Although aligned nanofibers have been shown to enhance neurite outgrowth, cell interactions with nanofibers embedded in a hydrogel matrix remain unexplored. In certain embodiments, single nanofibers supported by enzymatically degradable hydrogel in stable 3D architectures will accelerate axon growth and improve functional outcomes in vivo. The first step to testing this hypothesis was examining the interactions of individual cells with nanofiber-hydrogel composites in vitro. Specifically, the effects of nanofiber (diameter, spacing) and hydrogel (degradability) properties on alignment and axon elongation of neural cells embedded in nanofiber-hydrogel composites were investigated. This investigation was used to identify parameters that favor maximal neural cell alignment and directed axon elongation in vitro.

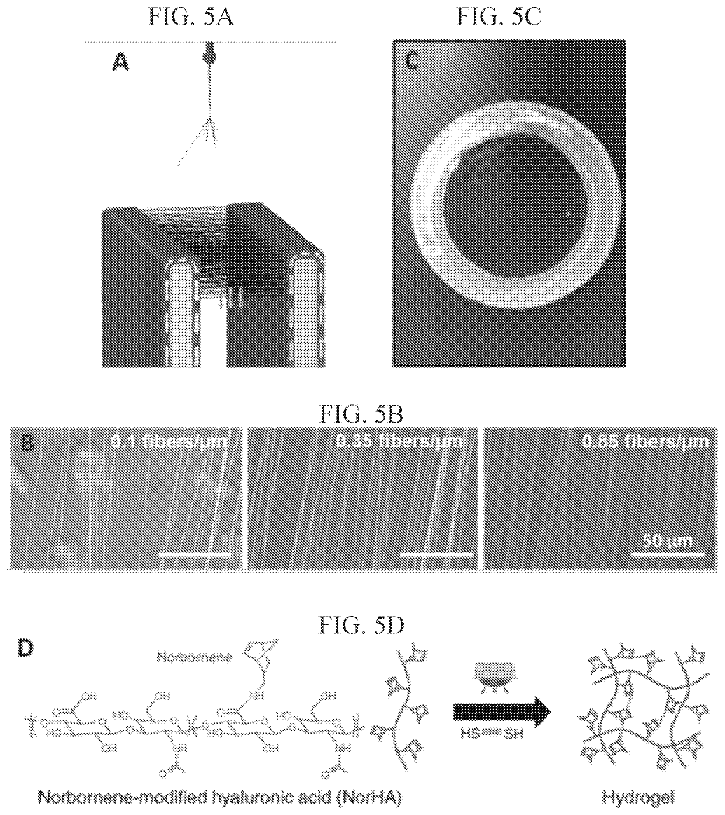

While a multitude of in vitro and in vivo studies have shown the potential of aligned nanofibers for axon regeneration, cellular responses to individual aligned nanofibers crosslinked within a 3D hydrogel environment have never been tested. This is largely due to technical challenges in fabricating and handling fragile unsupported nanofibers. To overcome this challenge, an automated track electrospinning technique allowed for the fabrication of highly abrication of highly aligned nanofiber arrays nanofibers and tunable lateral nanofiber spacing (FIGS. 5A and 5B). Single-layer nanofiber arrays were then encapsulated in a hydrogel by dip coating the aligned nanofiber arrays. It was also demonstrated that nanofiber-hydrogel composite films can be stacked atop one another to form thick cohesive solid constructs FIG. 5C). This layer-by-layer assembly technique leverages the thermoreversible properties of gelatin and light-mediated reactions that covalently crosslink the hydrogel matrix. For example, norbornene-modified macromers can undergo a light-mediated reaction with di-thiol crosslinkers to form hydrogels (FIG. 5D). When peptides with tunable enzymatic degradability (Deg) are used as the di-thiol crosslinker, the supporting hydrogel that anchors nanofibers in 3D space can be amenable to cell spreading.

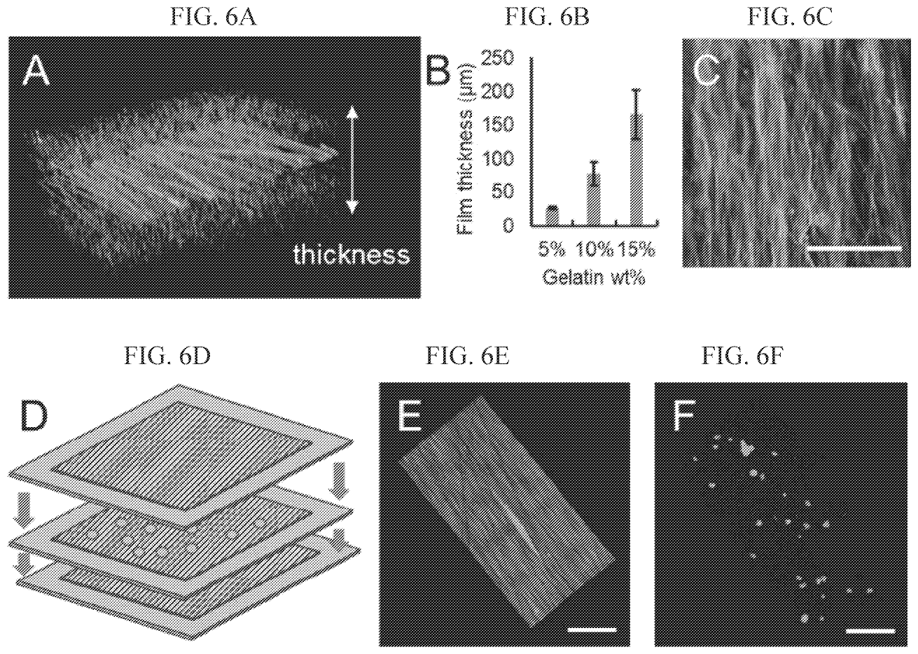

The data provided herein demonstrate that neural cells encapsulated in 3D nanofiber-hydrogel composites are viable and able to sense mechanical signals from embedded nanofibers. For these studies, PC-12 cells were chosen due to their ability to differentiate into ganglion neuron-like cells under neural growth factor (NGF) stimulation. NGF-treated (50 ng/ml) PC-12 cells were encapsulated within (3D) nanofiber-hydrogel composites or control gels without fibers. To visualize the nanofibers alone and in hydrogel composites, fibers were labeled with DiI (Biotium, 0.167% wt/wt) and hydrogels contained green fluorescent microspheres (Sigma, 1:1000) (FIG. 6A). Quantification of single layers showed an increase in thickness in a hydrogel-concentration manner, ranging from 25 μm to 160 μm for nanofibers embedded in gelatin hydrogels ranging from 5 to 15 wt %, respectively (FIG. 6B). It was demonstrated that human neural stem cells (NSCs) align in the direction of aligned nanofibers and stained positive for βIII tubulin (FIG. 6C). These promising results were done on 2D nanofiber sheets, and the nanofiber-composite design relies on the ability for neural cells to sense nanofibers and deform the hydrogel matrix in 3D. To test this approach, nanofibers were embedded in a methacrylated gelatin solution (GelMe) containing NGF-treated PC-12 cells. A three-layer construct was made, with acellular top and bottom layers FIG. 6D) and photopolymerized with ultraviolet light (5 mW/cm², 5 min). In as little as 24 hours, PC-12 cells were able to sense the nanofibers in 3D hydrogels, as evidenced by significant alignment and spreading in the direction of the nanofibers (FIG. 6E). This directional morphology was not evident in PC-12 cells encapsulated in 3D hydrogels without the presence of nanofiber guidance (FIG. 6F). This data shows that neural cells encapsulated in 3D nanofiber-hydrogel composites are viable and can spread within the gel to respond to mechanical cues from embedded aligned nanofibers.

This research helps identify governing relationships between nerve cell morphological behavior and material properties. Furthermore, the optimal nanofiber and hydrogel parameters for maximal neural cell axon extension and alignment are determined and the benefit of attached growth factor gradients on axon growth are assessed. This information helps determine the parameters for nanofiber-hydrogel composite nerve grafts that will be tested in vivo in a rat sciatic nerve gap model. If there are any issues culturing primary rat neurons in scaffolds, then immortalized neural cells line (PC-12) used in preliminary experiments are substituted. If challenges arise from functionalizing PLLA nanofibers with heparin at the surface concentrations desired, then an alternative di-NH₂-PEG based approach to attached heparin to PLLA nanofibers are explored. If heparin functionalized PLLA nanofibers do not have enough free thiol groups to adequately bind to the hydrogel matrix, the PLLA-SH fraction in the polymer solution are increased. If NGF gradients do not result in enhanced neurite growth, larger gradients (e.g., 0-25 ng/cm²) and alternate growth factors (e.g., VEGF, BDNF) can be easily tested.

Engineering Nanofiber-Hydrogel Synthetic Nerve Grafts

Robust implantable synthetic nerve conduits consisting of multiple nanofiber-hydrogel composite layers were constructed. Although not wishing to be limited by theory, in certain embodiments, nerve grafts with clinically viable geometry and stability can be constructed with a nanofiber-hydrogel film layer-by-layer approach and subsequent nanofiber conduit wrapping. To recapitulate the microarchitecture of native nerve, it is important to reduce nanofiber-hydrogel composite film thickness while maintaining alignment and structural stability. Layer thickness was optimized independent of gelatin concentration by tuning dip removal speed and solution temperature. An important clinical criterion of this synthetic graft is storage as an off-the-shelf product. Thus, graft microstructure, mechanics, and bioactivity were evaluated after sterile storage time of up to six months.

For clinical application, peripheral nerve grafts are required in a range of diameters (in the order of mm) and lengths (in the order of cm) to bridge disconnected nerve stumps, depending on location and severity. Further, commercially viable grafts must be able to be stored without compromising functionality. Therefore, the properties of nanofiber-hydrogel composite nerve grafts were evaluated for a period of up to six months. This section provides data directed to (1) investigating processes to optimize layer thickness, (2) scaling of layer-by-layer assembly approach to clinically relevant geometries, and (3) evaluating graft stability after storage up to 6 months based on geometry, microstructure, mechanics, and bioactivity.

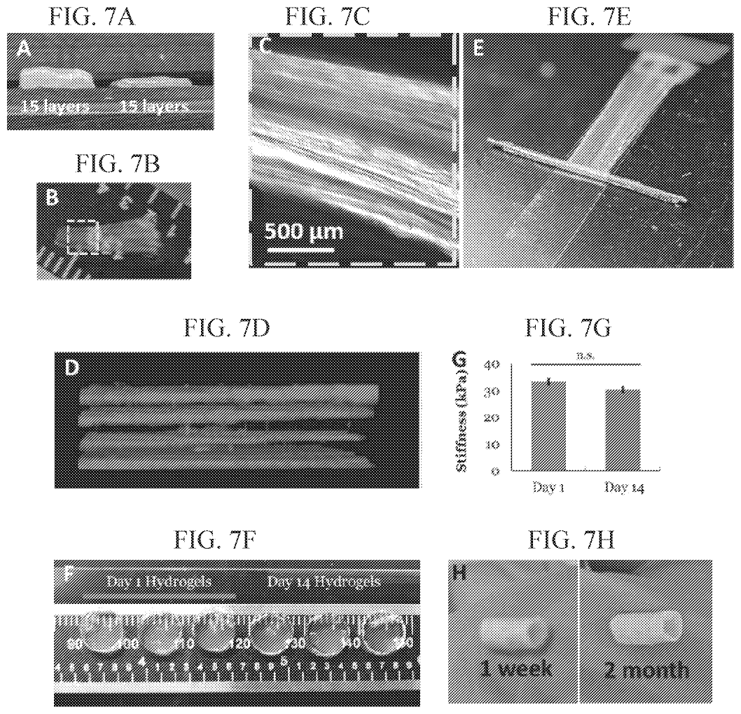

The data demonstrate that (1) nanofiber-hydrogel composite films can be stacked to form grafts of clinically relevant thickness and (2) the geometry and bulk stability of nanofiber-hydrogel grafts is conserved after long-term storage. Fiber density of layer-by-layer 3D grafts is dependent on layer thickness. Thus, preliminary experiments were performed to show the potential to control layer thickness via film withdrawal speed or dipping temperature (FIG. 7A). Dip coating in 5% GelMe at 37° C. with a fast (˜50 mm/s) withdrawal speed produced a nanofiber-hydrogel stack large enough to construct a rat sciatic nerve graft (23 layers, ˜2 mm thick, FIGS. 7B-7C) with distinct spacing between the layers (FIG. 7D). To enclose the nanofiber-hydrogel stacks, a robust aligned nanofiber outer conduit was wrapped around nanofiber-hydrogel composites, via a custom wrapping procedure (FIG. 7E). The outer conduit results in increased structural stability and forms a readily implantable graft with strong mechanical attachment between the outer conduit and inner hydrogel composite.

Thick GelMe hydrogels tracked over 14 days in PBS at 4° C. (FIG. 7F) showed that crosslinked gelatin hydrogels do not exhibit significant swelling behaviors that could stress the confined hydrogel composite and disrupt internal nanofiber architecture. Further, this preliminary study showed that hydrogels do not experience a significant reduction in compressive modulus due to hydrolytic degradation at 14 days (FIG. 7G). Indeed, grafts fabricated in preliminary experiments were stable by visual inspection when stored in sterile PBS for 2 months (FIG. 7H). This preliminary data showed that nanofiber-hydrogel composite grafts can be fabricated with clinically relevant dimensions and wrapped in a robust outer conduit to form implantable grafts. Grafts retain their shape and structural stability after at least two months, which makes them promising for clinical applications. In certain embodiments, the disclosed grafts demonstrate good stability in PBS for up to six months and retain bioactivity which will be confirmed by cell penetration, neural cell alignment, and guided axon growth. If significant cell infiltration is not observed, additional nanofiber-hydrogel composite (higher layer thickness) and hydrogel (lower wt %) parameters can be tested. If grafts are not stable or do not retain their microarchitecture, macromers less prone to degradation (e.g., hyaluronic acid) can be evaluated. The grafts that are found to have superior microstructure, mechanics, and bioactivity will be tested in a rat sciatic nerve gap model.

Test Efficacy of Synthetic Nerve Grafts in a Rat Sciatic Nerve Gap Model

Early reports identified structural similarities between human and rat sciatic nerves. As such, the rat sciatic nerve is by far the most frequently used animal model for studying nerve repair strategies including gap models. In certain embodiments, synthetic nerve grafts with individual aligned nanofibers arranged in a stable 3D architecture within a permissive hydrogel matrix provide directional physical cues that direct and accelerate axon growth and improve gait and electrophysiological outcomes in vivo.

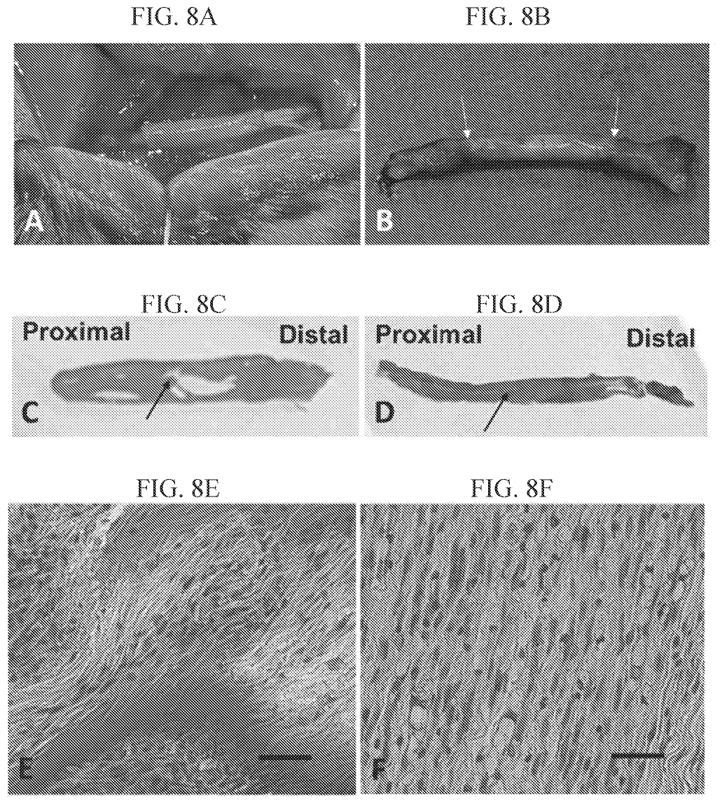

A pilot study was performed to assess the suitability of proposed grafts for surgical implantation in a rat sciatic nerve model. Ten grafts (5% GelMe, 50 layers) were implanted into mixed male & female Lewis rats with the following groups: (1) nanofiber-hydrogel synthetic grafts wrapped in nanofiber conduit (n=4), (2) control grafts in (1) without nanofibers (n=5) and (3) allograft (n=1). Rats were euthanized at 1, 2, 3 and 4 weeks, sectioned longitudinally in paraffin and stained with H&E for morphology. Grafts handled well surgically and integrated well with the sciatic nerve (FIGS. 8A-8B). H&E histology of nanofiber-hydrogel synthetic grafts showed aligned tissue growth extending longitudinally to connect the distal and proximal nerve stumps as soon as 1 week. By week 4, robust aligned tissue growth filled nearly the entire conduit (FIG. 8C). H&E images of a nanofiber-hydrogel graft are compared to allograft at the 4 week time point in FIGS. 8C-8F. Histological processing, imaging and analysis of data generated from this extremely recent study is ongoing. This preliminary data showed that (1) nanofiber-hydrogel grafts are implantable and (2) provided strong evidence of the ability of nanofiber-hydrogel materials to support organized nerve ingrowth and nerve regeneration in vivo.

The rat sciatic gap model has been used extensively in the literature to assess axon regeneration and functional performance of empty nerve conduits, and conduits filled with hydrogel or folded dense nanofiber mats. The preliminary data shows that the materials disclosed herein can be used as synthetic grafts to bridge rat sciatic nerve gaps. The results prove that the proposed graft can be implanted and integrates well in the rat sciatic nerve model. Histology provides evidence that synthetic grafts can support tissue ingrowth and nerve regeneration. The sciatic nerve can be transected, and the proximal and distal nerve stumps can be bridged with engineered nerve grafts and appropriate controls. Functional outcomes (gait, electrophysiology), histology of harvested nerve grafts (axon length, count, diameter, myelination), and innervated muscle atrophy can be assessed to evaluate the clinical potential of implanted nerve grafts. The in vivo environment may degrade the nanofiber-hydrogel composite grafts more quickly than the in vitro environment. If premature degradation is observed, then stable non-peptide based crosslinkers (e.g., dithiothreitol, DTT) and hyaluronic acid-based macromers (i.e., HANor) can be developed. If additional behavioral, anatomical, electrophysiology or histology outcomes such as tetanic muscle force or retrograde tracing appear beneficial to distinguish graft performance, they can be added to the study.

### Example 2: Creating Aligned Polycaprolactone Nanofiber Hydrogel Composites Through Layer-by-Layer Assembly

Methods

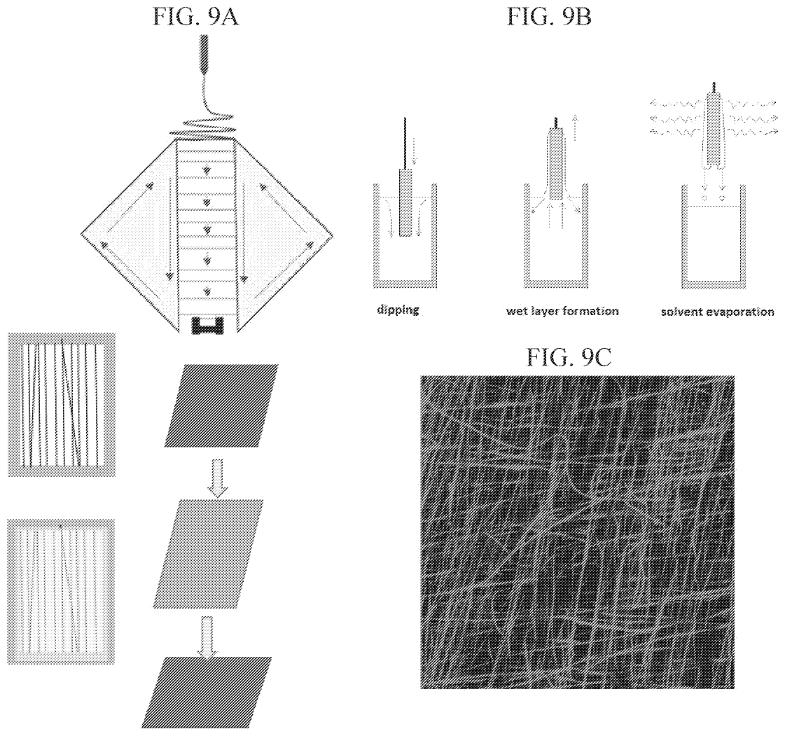

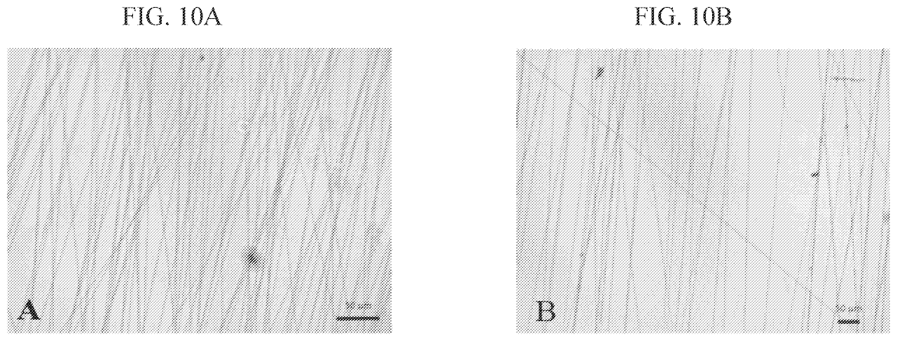

An 18 wt % polycaprolactone solution containing a 0.2 wt % Nile red dye was spun utilizing a custom electrospinner within the lab (FIGS. 9A-9C). The spin time can be used to control the density of the fibers (FIGS. 10A-10B). The fibers were collected on a tray and then mounted on thin plastic frames to stabilize the fibers. Fibers were then dip coated in different concentrations of gelatin methacrylate (GelMe), varying from 2.5% to 10% (FIG. 11). To create the multilayer hydrogels, single thin films were stacked on top of each other to create five-layer hydrogels where layers were at alternating temperatures of 20° C. (solid) and 37° C. (liquid). These multilayer hydrogels were then crosslinked into a thick cohesive structure by a UV lamp for 10 minutes. Final structures were imaged using a confocal microscope to visualize the nanofiber architecture within the hydrogel matrix.

Results and Selected Discussion

Tissue engineering grafts are needed for the use of regenerating aligned soft tissues. Some of these tissues are nerve, spinal cord and skeletal, smooth, and cardiac muscle. Utilizing aligned nanofibers in the grafts enhances the alignment and elongation of the cells. However, aligned nanofibers are generally tested as 2D films only, which is not appropriate for 3D tissue regeneration. The 2D films do not have the proper geometry to act as they would in the body and dense fiber packing in the films blocks cell infiltration.

The present disclosure provides continuous aligned nanofibers that are embedded in a hydrogel to achieve the desired properties such as cell permeability and tunable aligned fiber spacing. A layer by layer assembly approach was used with thin thermo-reversible UV crosslinkable hydrogel films to create a 3D cohesive structure with tunable spacing between aligned fiber arrays. The 3D cohesive structures were studied to determine the relationships between processing parameters and 3D nanofiber architecture and to investigate cells' ability to align within these composites.

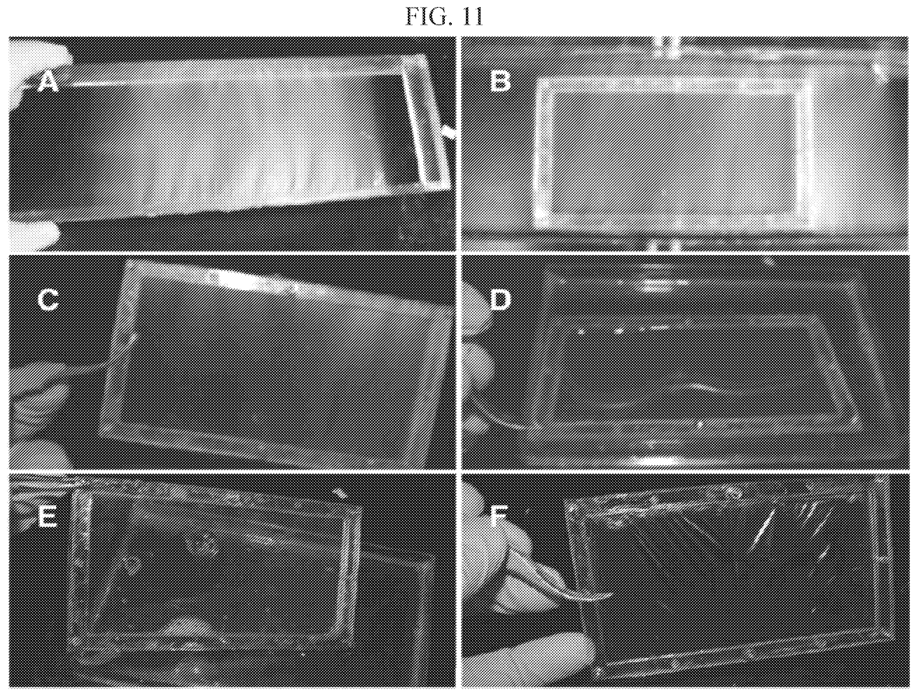

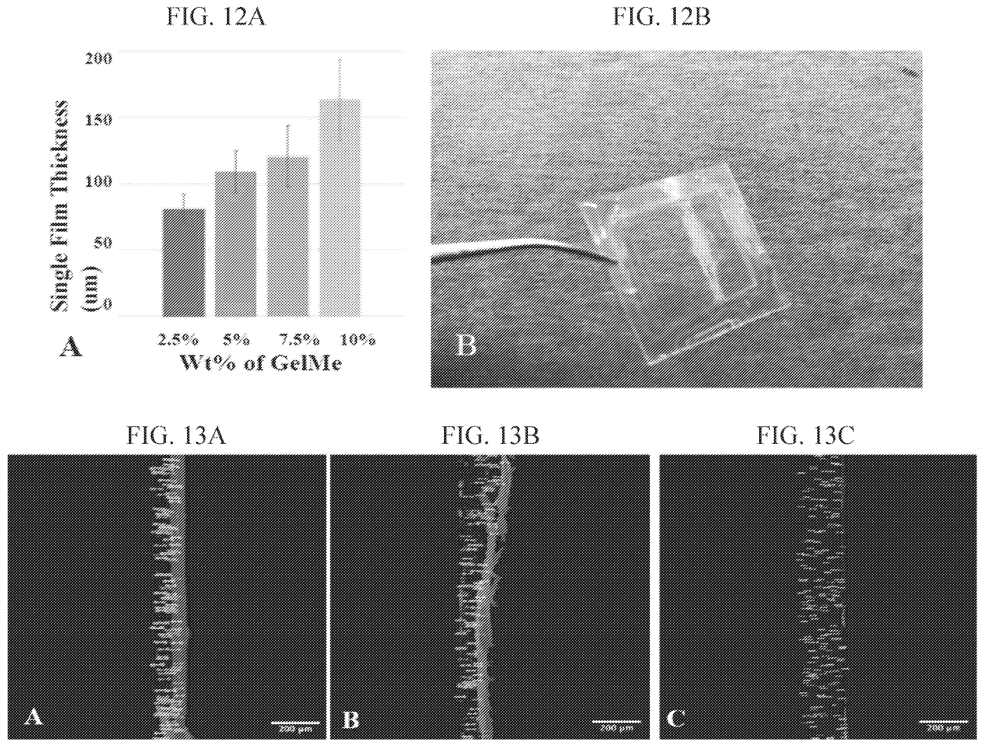

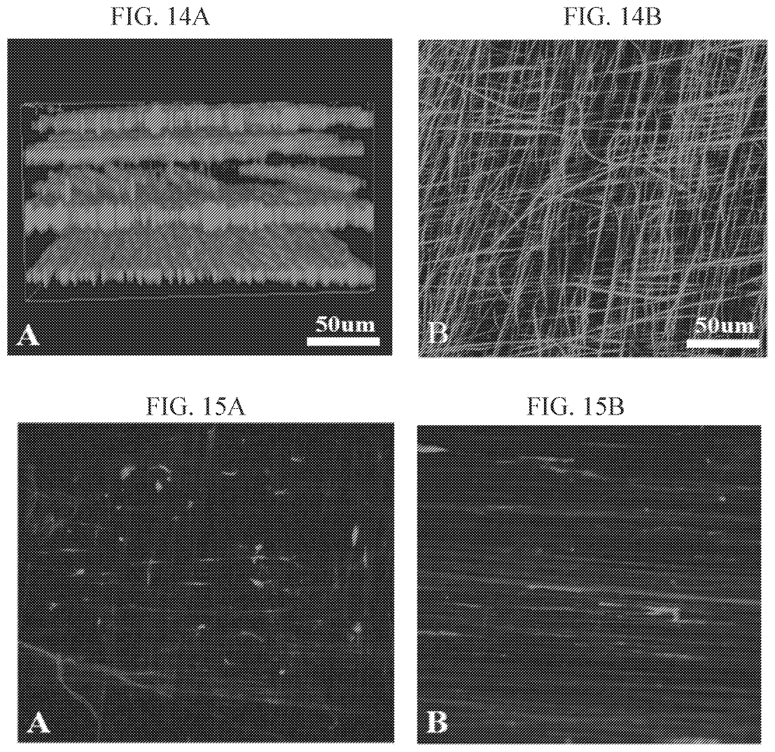

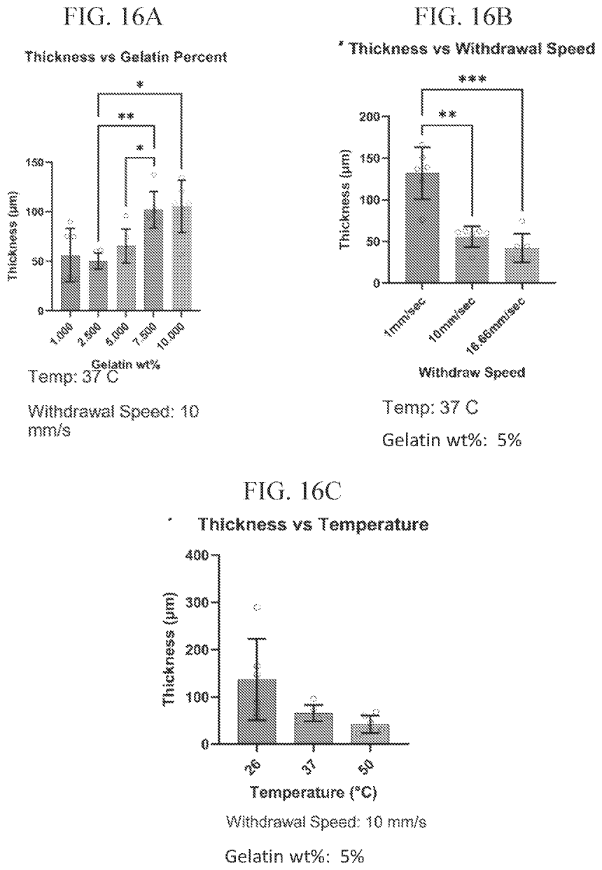

The thickness of single nanofiber/hydrogel composite films was found to be between 83 m and 160 m. The film thickness was found to be dependent on the concentration of the gelatin solution (FIGS. 12A-12B; FIGS. 13A-13C). As the films were placed into the stacking process, it was observed that the lower layers had a decreasing film thickness (FIGS. 14A-14B). This came from the added weight being placed on as each layer was added. Cells were then seeded within the middle layer of the hydrogel. It was observed that the cells began to attach to the nanofibers within the first day (FIG. 15A). From there, the cells began to elongate along the fibers and by day 4, the cells had elongated a great distance over the nanofiber (FIG. 15B).

The confocal image of the layered hydrogel shows that it is possible to create a cohesive aligned nanofiber hydrogel composite with varying thicknesses. Cells are able to align and elongate along the fibers. Layer thickness can be controlled by modifying hydrogel solution concentration, therefore allowing for closer z-packing and better control of fiber spacing and architecture.

### Example 3: Modifying Process Parameters to Tune the Thickness of a Hydrogel/Nanofiber Film

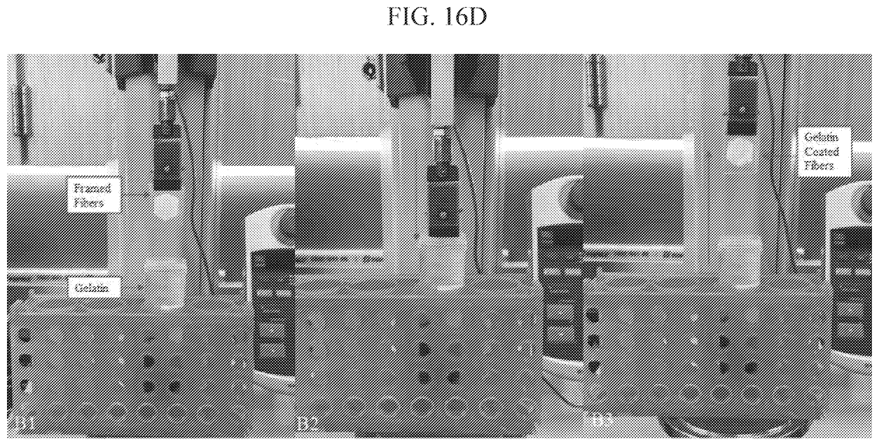

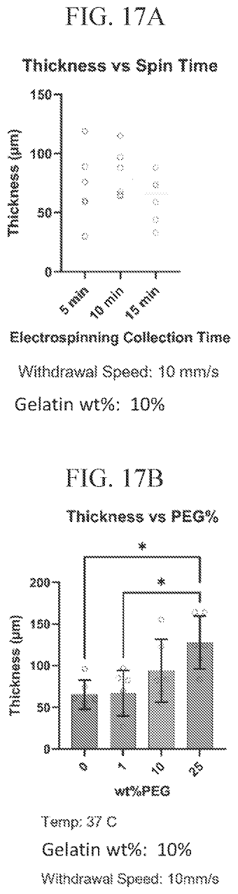

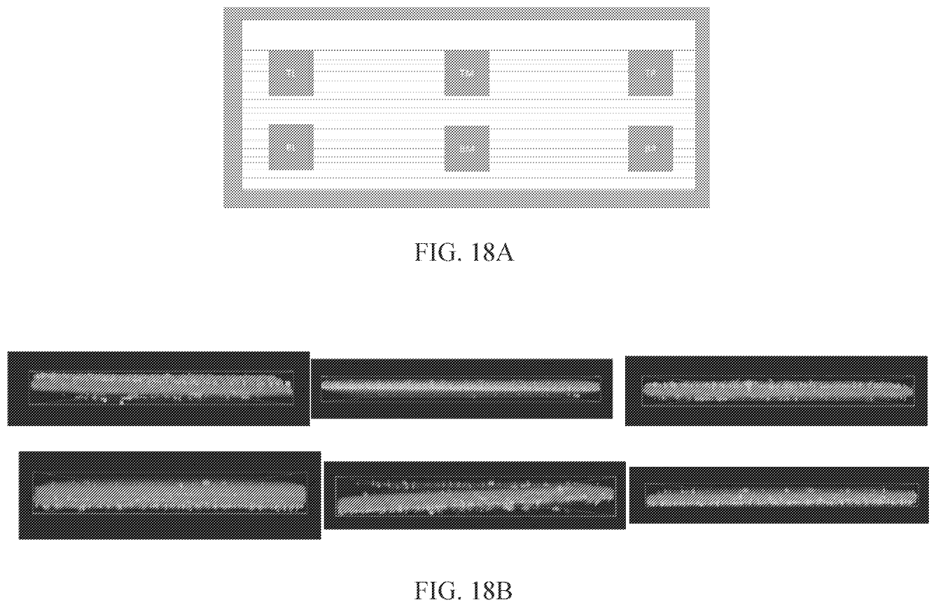

Gelatin/Polycaprolactone (PCL) nanofiber composite hydrogel films were assembled as follows. Aligned nanofiber arrays with a linear fiber density of ˜0.5 fibers per micron width were created using an automated track device. The films were immersed in a gelatin solution (1-10% wt/v) held at temperatures from 25-50° C. A mechanical testing machine was used to control the withdrawal speeds between 1-16.66 mm/s. (FIGS. 16A-16D) Composite film thickness was measured on optical images taken perpendicular to the thickness of the final film. Six replicates were tested per group. Results showed that film thickness is dependent on gelatin solution wt/v percentage and temperature (which both affect solution viscosity) and withdrawal speed. Further experiments tested the effect of fiber density and of blending 1-25 wt % methacrylated polyethylene glycol (PEG-Me) into the PCL nanofibers (FIGS. 17A-17B). As hydrophilicity increased with increasing PEG-Me composition, the thickness of the films increased. The thickness and uniformity of films can be confirmed using confocal microscopy when a fluorescent tag was incorporated into a gelatin methacrylate (Gel-Me) molecule used with an identical dipping procedure (FIGS. 18A-18B).

### Example 4: Mechanical Testing to Confirm Layer-Layer Intermixing/Interdigitation

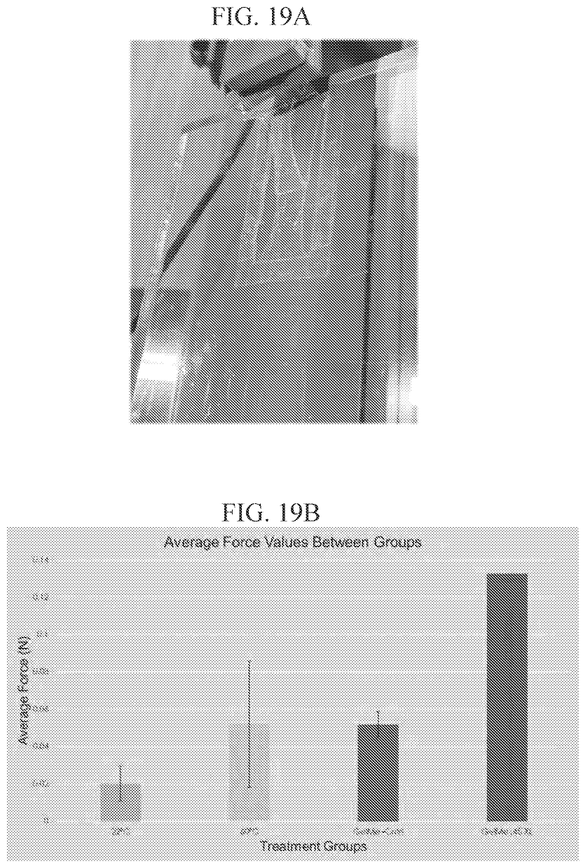

A modified peeling adhesion test was performed to confirm that adhesion between composite layers is enhanced by liquid-semisolid material characteristics utilized during layer-by-layer stacking. Here an increase in average force during peeling indicates increased intermixing/interdigitation of layers. Tests were performed to assess the impact of temperature on gelatin layer intermixing and UV crosslinking time on Gel-Me layer intermixing. For gelatin, a base layer was dipped in 5% gelatin solution at 60° C., then allowed to cool to room temperature. For the control group a second layer was dipped and cooled in the same way before stacking. For the test group, the second layer was dip coated at 60° C. and added to the base layer without allowing time to cool. It was hypothesized that this would partially liquefy the underlying layer and promote mixing and interdigitation between the two layers. For Gel-Me, a base layer was dipped in 5% Gel-Me solution at 60° C. and exposed to UV light at 10 mW/cm² for 3 minutes to crosslink. For the control group, a second layer was dipped and crosslinked in the same way before stacking. For the test group, the base layer was crosslinked for only 45 seconds. Then a second layer was dipped in 5% Gel-Me solution at 60° C. and immediately added on top of the base layer, followed by UV crosslinking the stacked structure for an additional 45 seconds. It was hypothesized that less UV crosslinking prior to stacking would promote layer interdigitation. The plastic frames holding the two films were pulled apart using a mechanical testing machine while the force was measured. An increase in the peeling force was observed for both test groups versus control. This indicated enhanced layer interdigitation with temperature and UV regulated liquid/solid phase control during stacking (FIGS. 19A-19B).

### Example 5: Tuning Layer Thickness Via Mechanical Compression During Stacking

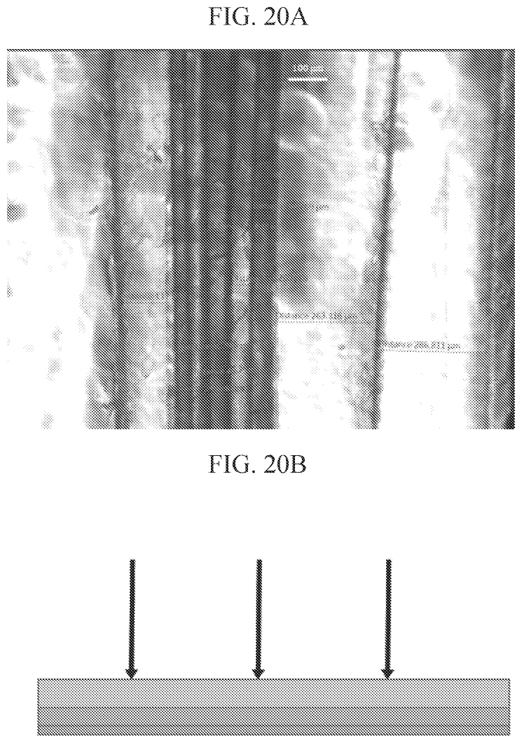

PCL nanofiber arrays were dipped in 5% Gel-Me solution to form films and sequentially stacked on top of one another with 45 seconds of UV crosslinking applied after each layer was added. Some mechanical force was applied to the structure as each layer was placed on the construct. It was observed that the layers in the final structures appeared to be thinner at the bottom of the stack (FIGS. 20A-20B). It is hypothesized that this is due to compression of the gels due to the external mechanical force applied to incompletely crosslinked layers. This observation indicates the possibility of modulating layer thickness using mechanical compression in conjunction with controlled gelation and crosslinking.

### Example 6: Cell Infiltration into Thick Multilayer Composites

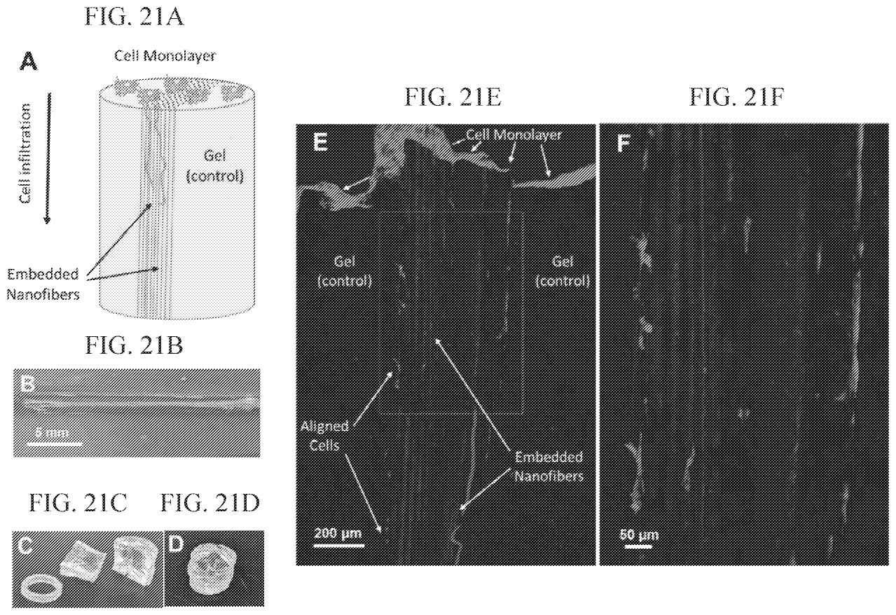

PCL nanofiber arrays were dipped in 5% Gel-Me solution to form films and sequentially stacked on top of one another with 1 minute of UV crosslinking applied after each layer was added. Ten layers were constructed, and then additional Gel-Me solution (without fibers) was crosslinked to the top and bottom of the stack to form a construct (FIG. 21). A rod-like segment of the construct was held vertically by a PDMS holder (FIGS. 21A and 21C-21D) and seeded with cells on the surface of one of its ends. After 14 days, constructs were fixed in formalin and stained for nuclei and actin. PCL fibers were stained blue. Confocal microscopy was used to visualize a cell monolayer formed on the seeded surface of the construct and to view cells infiltrating down into the volume of the construct (FIGS. 21E-21F). It was observed that cells migrated deep into the constructs in the areas where aligned nanofibers were present, but did not infiltrate into areas without fibers. It is hypothesized that the embedded nanofibers encourage cell infiltration along the length of the fibers via physical guidance cues.

### Example 7: Incorporating Gradients onto/into Nanofiber and/or Hydrogel

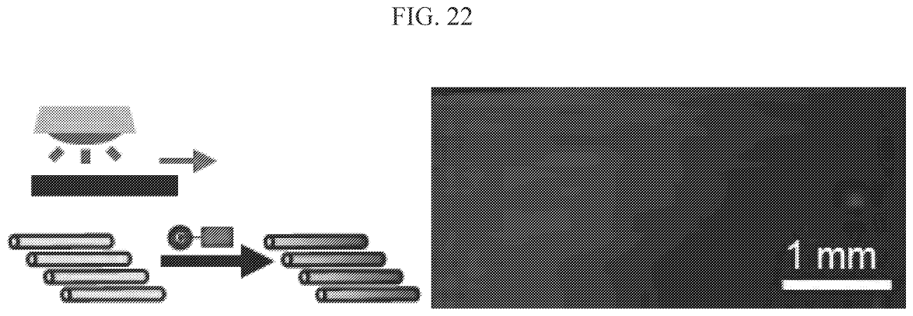

The polymer nanofiber surface was decorated with a peptide gradient using the following procedure. Eight-arm polyethylene glycol terminated with norbornene (PEG-Nor) was added to PCL solution prior to electrospinning at 10% wt/wt. Peptides with a thiol group at one end and a florescent rhodamine group at the other were synthesized with a peptide synthesizer. The thiol on the peptide was covalently crosslinked to norbornene on the blended PCL-(PEG-Nor) nanofiber using a UV light mediated Michael addition reaction in the presence of a photoinitiator. The quantity of peptide binding is proportional to the UV light exposure time, so a gradient was made by using a sliding mask approach (FIG. 22). After UV crosslinking the nanofiber arrays were washed to remove unbound peptide. The increasing intensity of fluorescence as measured by confocal microscopy indicates a gradient of increasing concentration of peptide on the nanofiber surface (FIG. 22). A similar masking approach can be used to construct a hydrogel with a gradient ratio of highly degradable vs. less degradable linking molecules. In this way a hydrogel matrix can be made which will have a variable degradation rate along its length that increases from one end to the other.

### Enumerated Embodiments

In some aspects, the instant specification is directed to the following non-limiting embodiments:

Embodiment 1: A method of regenerating an aligned soft tissue in a subject, the method comprising surgically implanting a synthetic tissue graft in the subject at a site of missing or injured soft tissue, wherein the synthetic tissue graft is a multi-layer composite comprising at least a first and a second sheets of aligned nanofibers coated with a biodegradable hydrogel, wherein the at least first and second sheets of coated aligned nanofibers are stacked directly on top of each other, and wherein a portion of the biodegradable hydrogel coating on the first sheet is mixed and crosslinked with a portion of the biodegradable hydrogel coating on the second sheet.

Embodiment 2: The method of Embodiment 1, wherein the aligned soft tissue is a nerve and the synthetic tissue graft is a synthetic nerve graft.

Embodiment 3: The method of Embodiment 2, wherein the synthetic nerve graft comprises the multi-layer composite formed into a cylinder shape, wherein the outside surface of the cylinder is covered with an outer conduit.

Embodiment 4: The method of any one of Embodiments 1-3, wherein each of the at least first and second sheets of coated aligned nanofibers independently comprise substantially aligned individual nanofibers having a controlled diameter and spacing.

Embodiment 5: The method of any one of Embodiments 1-4, wherein each of the at least first and second sheets of coated aligned nanofibers independently comprise substantially aligned individual nanofibers having a length of about 0.25 cm to about 30 cm.

Embodiment 6: The method of any one of Embodiments 1-5, wherein each of the at least first and second sheets of coated aligned nanofibers independently comprise substantially aligned individual nanofibers that are covalently bonded to the biodegradable hydrogel coating.

Embodiment 7: The method of any one of Embodiments 1-6, wherein the at least first and second sheets of coated aligned nanofibers comprise substantially aligned individual polycaprolactone, alginate, polyacrylonitrile, or poly(lactic acid) nanofibers.

Embodiment 8: The method of Embodiment 4, wherein each of the at least first and second sheets of aligned nanofibers coated with a biodegradable hydrogel has a controlled thickness and the multi-layer composite has a defined three dimensional structure.

Embodiment 9: The method of any one of Embodiments 1-8, wherein the biodegradable hydrogel coating comprises gelatin, poly(ethylene glycol), hyaluronic acid, collagen, polyacrylamide, alginate, or chemically modified versions thereof.

Embodiment 10: The method of any one of Embodiments 1-9, wherein the biodegradable hydrogel coating comprises gelatin or chemically modified versions thereof.

Embodiment 11: The method of any one of Embodiments 1-10, wherein cells at the site of the missing or injured tissue degrade the biodegradable hydrogel and grow into the multi-layer composite in the direction of the at least first and second sheets of coated aligned nanofibers, thus regenerating the aligned soft tissue in the subject.

Embodiment 12: The method of any one of Embodiments 1-11, wherein the biodegradable hydrogel coating is reversibly or irreversibly gelled or cured before, during or after layer stacking.

Embodiment 13: The method of any one of Embodiments 1-12, wherein the multi-layer composite comprises one or more additional sheets of aligned nanofibers coated with a biodegradable hydrogel such that the multi-layer composite comprises three or more sheets of coated aligned nanofibers stacked on top of each other, wherein each coated sheet of aligned nanofibers completely covers an adjacent sheet of coated aligned nanofibers, and wherein a portion of the biodegradable hydrogel coating on each sheet of coated aligned nanofibers is mixed and crosslinked with a portion of the biodegradable hydrogel coating on directly adjacent coated sheets of aligned nanofibers.

Embodiment 14: The method of any one of Embodiments 1-13, further comprising forming a gradient of a chemical modification on the aligned nanofibers or in the coated biodegradable hydrogel in the direction of the nanofibers.

Embodiment 15: The method of Embodiment 14, wherein the chemical modification comprises a peptide tethered to a surface of the nanofibers or to a molecular backbone of the biodegradable hydrogel, or a degradable crosslinker in the biodegradable hydrogel.

Embodiment 16: A synthetic nerve graft comprising a multi-layer composite, the multi-layer composite comprising at least a first and second sheets of aligned nanofibers coated with a biodegradable hydrogel, wherein the at least first and second sheets of coated aligned nanofibers are stacked directly on top of each other, and a portion of the biodegradable hydrogel coating on the first sheet is mixed and crosslinked with a portion of the biodegradable hydrogel coating on the second sheet.

Embodiment 17: The synthetic nerve graft of Embodiment 16, wherein the synthetic nerve graft further comprises an outer conduit covering the outside surface of a cylinder-shaped multi-layer composite.

Embodiment 18: The synthetic nerve graft of Embodiment 16 or 17, wherein each of the at least first and second sheets of coated aligned nanofibers independently comprise substantially aligned individual nanofibers having a controlled diameter and spacing.

Embodiment 19: The synthetic nerve graft of any one of Embodiments 16-17, wherein each of the at least first and second sheets of coated aligned nanofibers independently comprise substantially aligned individual nanofibers having a length of about 0.25 cm to about 30 cm.

Embodiment 20: The synthetic nerve graft of any one of Embodiments 16-19, wherein each of the at least first and second sheets of coated aligned nanofibers independently comprise substantially aligned individual nanofibers that are covalently bonded to the biodegradable hydrogel coating.

Embodiment 21: The synthetic nerve graft of any one of Embodiments 16-20, wherein the at least first and second sheets of coated aligned nanofibers comprise substantially aligned individual polycaprolactone, alginate, polyacrylonitrile, or poly(lactic acid) nanofibers.

Embodiment 22: The synthetic nerve graft of Embodiment 18, wherein each of the at least first and second sheets of aligned nanofibers coated with a biodegradable hydrogel has a controlled thickness and the multi-layer composite has a defined three dimensional structure.

Embodiment 23: The synthetic nerve graft of any one of Embodiments 16-22, wherein the biodegradable hydrogel coating comprises gelatin, poly(ethylene glycol), hyaluronic acid, collagen, polyacrylamide, alginate, or chemically modified versions thereof.

Embodiment 24: The synthetic nerve graft of any one of Embodiments 16-23, wherein the biodegradable hydrogel coating comprises gelatin or chemically modified versions thereof.

Embodiment 25: The synthetic nerve graft of any one of Embodiments 16-24, wherein the multi-layer composite comprises one or more additional sheets of aligned nanofibers coated with a biodegradable hydrogel such that the multi-layer composite comprises three or more sheets of coated aligned nanofibers stacked on top of each other, wherein each coated sheet of aligned nanofibers completely covers an adjacent sheet of coated aligned nanofibers, and wherein a portion of the biodegradable hydrogel coating on each sheet of coated aligned nanofibers is mixed and crosslinked with a portion of the biodegradable hydrogel coating on directly adjacent coated sheets of aligned nanofibers.

Embodiment 26: The synthetic nerve graft of any one of Embodiments 16-25, wherein the synthetic nerve graft bridges a nerve gap in a subject and promotes neuron alignment and axon elongation across the nerve gap.

Embodiment 27: The synthetic nerve graft of any one of Embodiments 16-26, wherein the aligned nanofibers or the coated biodegradable hydrogel has a gradient of a chemical modification in the direction of the nanofibers.

Embodiment 28: The synthetic nerve graft of Embodiment 27, wherein the chemical modification comprises a peptide tethered to a surface of the nanofibers or to a molecular backbone of the biodegradable hydrogel, or a degradable crosslinker in the biodegradable hydrogel.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of regenerating an aligned soft tissue in a subject, the method comprising surgically implanting a synthetic tissue graft in the subject at a site of missing or injured soft tissue,
wherein the synthetic tissue graft is a multi-layer composite comprising at least a first and a second sheets of aligned nanofibers coated with a biodegradable hydrogel,
wherein the at least first and second sheets of coated aligned nanofibers are stacked directly on top of each other,
and
wherein a portion of the biodegradable hydrogel coating on the first sheet is mixed and crosslinked with a portion of the biodegradable hydrogel coating on the second sheet.

2. The method of claim 1, wherein the aligned soft tissue is a nerve and the synthetic tissue graft is a synthetic nerve graft.

3. (canceled)

4. The method of claim 1, wherein each of the at least first and second sheets of coated aligned nanofibers independently comprise substantially aligned individual nanofibers having a controlled diameter and spacing.

5. (canceled)

6. The method of claim 1, wherein each of the at least first and second sheets of coated aligned nanofibers independently comprise substantially aligned individual nanofibers that are covalently bonded to the biodegradable hydrogel coating.

7. The method of claim 1, wherein the at least first and second sheets of coated aligned nanofibers comprise substantially aligned individual polycaprolactone, alginate, polyacrylonitrile, or poly(lactic acid) nanofibers.

8. (canceled)

9. The method of claim 1, wherein the biodegradable hydrogel coating comprises gelatin, poly(ethylene glycol), hyaluronic acid, collagen, polyacrylamide, alginate, or chemically modified versions thereof.

10. The method of claim 1, wherein the biodegradable hydrogel coating comprises gelatin or chemically modified versions thereof.

11. The method of claim 1, wherein cells at the site of the missing or injured tissue degrade the biodegradable hydrogel and grow into the multi-layer composite in the direction of the at least first and second sheets of coated aligned nanofibers, thus regenerating the aligned soft tissue in the subject.

12. (canceled)

13. The method of claim 1, wherein the multi-layer composite comprises one or more additional sheets of aligned nanofibers coated with a biodegradable hydrogel such that the multi-layer composite comprises three or more sheets of coated aligned nanofibers stacked on top of each other,
wherein each coated sheet of aligned nanofibers completely covers an adjacent sheet of coated aligned nanofibers, and
wherein a portion of the biodegradable hydrogel coating on each sheet of coated aligned nanofibers is mixed and crosslinked with a portion of the biodegradable hydrogel coating on directly adjacent coated sheets of aligned nanofibers.

14. The method of claim 1, further comprising forming a gradient of a chemical modification on the aligned nanofibers or in the coated biodegradable hydrogel in the direction of the nanofibers.

15. (canceled)

16. A synthetic nerve graft comprising a multi-layer composite, the multi-layer composite comprising at least a first and second sheets of aligned nanofibers coated with a biodegradable hydrogel,
wherein the at least first and second sheets of coated aligned nanofibers are stacked directly on top of each other, and
a portion of the biodegradable hydrogel coating on the first sheet is mixed and crosslinked with a portion of the biodegradable hydrogel coating on the second sheet.

17. The synthetic nerve graft of claim 16, wherein the synthetic nerve graft further comprises an outer conduit covering the outside surface of a cylinder-shaped multi-layer composite.

18. The synthetic nerve graft of claim 16, wherein each of the at least first and second sheets of coated aligned nanofibers independently comprise substantially aligned individual nanofibers having a controlled diameter and spacing.

19. (canceled)

20. The synthetic nerve graft of claim 16, wherein each of the at least first and second sheets of coated aligned nanofibers independently comprise substantially aligned individual nanofibers that are covalently bonded to the biodegradable hydrogel coating.

21. The synthetic nerve graft of claim 16, wherein the at least first and second sheets of coated aligned nanofibers comprise substantially aligned individual polycaprolactone, alginate, polyacrylonitrile, or poly(lactic acid) nanofibers.

22. (canceled)

23. The synthetic nerve graft of claim 16, wherein the biodegradable hydrogel coating comprises gelatin, poly(ethylene glycol), hyaluronic acid, collagen, polyacrylamide, alginate, or chemically modified versions thereof.

24. The synthetic nerve graft of claim 16, wherein the biodegradable hydrogel coating comprises gelatin or chemically modified versions thereof.

25. The synthetic nerve graft of claim 16, wherein the multi-layer composite comprises one or more additional sheets of aligned nanofibers coated with a biodegradable hydrogel such that the multi-layer composite comprises three or more sheets of coated aligned nanofibers stacked on top of each other,
wherein each coated sheet of aligned nanofibers completely covers an adjacent sheet of coated aligned nanofibers, and
wherein a portion of the biodegradable hydrogel coating on each sheet of coated aligned nanofibers is mixed and crosslinked with a portion of the biodegradable hydrogel coating on directly adjacent coated sheets of aligned nanofibers.

26. The synthetic nerve graft of claim 16, wherein the synthetic nerve graft bridges a nerve gap in a subject and promotes neuron alignment and axon elongation across the nerve gap.

27. The synthetic nerve graft of claim 16, wherein the aligned nanofibers or the coated biodegradable hydrogel has a gradient of a chemical modification in the direction of the nanofibers.

28. (canceled)
